(19) United States
(12) Patent Application Publication
(10) Pub. No.: US 2019/0345513 A1
(43) Pub. Date: Nov. 14, 2019

(54) Stacking of Insecticidal and Herbicide Resistant Triple Gene [Kalgin-4] Expression in Plant

(71) Applicant: FB Genetics (Pvt) Limited, Lahore (PK)

(72) Inventors: Daniyal Jawed Qureshi, Lahore Cantt (PK); Hamza Nadeem Qureshi, Lahore Cantt (PK)

(21) Appl. No.: 16/407,938

(22) Filed: May 9, 2019

(30) Foreign Application Priority Data
May 9, 2018 (PK) 333/2018

(51) Int. Cl. C12N 15/82; C07K 14/32; C07K 14/195

(52) U.S. Cl. CPC C12N 15/8286; C07K 14/195; C07K 14/32; C12N 15/8274

(57) ABSTRACT

Recombinant or synthetic polynucleotide sequences comprising one Re-PAT gene identified as SEQ ID NO: 11 and two B. thuringiensis δ-endotoxin genes including Cry10A gene identified as SEQ ID NO: 12 and Cry11a12 gene identified as SEQ ID NO: 13, transformed in a mono/dicot plant, that are encoded to form a herbicidal and insecticidal toxin proteins in a transgenic plant, resulting in decreased resistance development against insecticidal toxins proteins and increased efficacy against the insect mortality, particularly pink bollworms and army worms. A method or an assay for detecting the presence of transgenic event Kalgin-4 based on the DNA sequence of the recombinant polynucleotide construct inserted into the genome of the transgenic plant and the genomic sequences flanking the insertion site.

### CROSS-REFERENCE TO RELATED APPLICATIONS

This application claims the benefit of Pakistani Application NO: PK 333/2018 filed 9 May 2018 the entire contents and substance of which is hereby incorporated by reference.

### SEQUENCE LISTING

The instant application contains a Sequence Listing which has been submitted electronically in ASCII format and is hereby incorporated by reference in its entirety. Said ASCII copy, created on May 9, 2019, is named FBG2_SL.txt and is 59,139 bytes in size.

### BACKGROUND OF THE INVENTION

### 1. Technical Field

The present invention relates to the field of genetic engineering of mono and/or dicot plants, more specifically the invention relates to the enhanced expression of synthetic insecticidal genes including Re-PAT and B. thuringiensis endotoxins Cry10A and Cry11a12 in transgenic plant specifically cotton plant (Gossypium hirsutum). The invention also relates to the plant, plant part, plant seeds and/or plant cells related to event Kalgin-4 and provide nucleotide molecules that are unique to the event and created in connection with insertion of transgenic DNA into the genome of a cotton plant and to assay for detecting the presence of Kalgin-4 event in the transgenic cotton plant sample.

### 2. Description of Related Art

Cotton is the most influential economic booster and cash crop, essential source of raw material to the textile enables the textile industry to survive and expand its volume. Cotton contributes 1% to GDP and has share of 5.1% in agriculture value additions. Massive decline in production of cotton was observed this year, and to maintain continuous supply to textile industry, raw cotton was imported during July which has increased to 345.363 thousand tonnes compared to last year during same period which was 97.354 thousand tonnes, so a growth of 254.75% was noticed while in value terms it touched to US$588.236 million against US$ 224.647 million showing a growth of 161.85%. Cotton was sown on a territory of 2917 thousand hectares, demonstrating a decline of 1.5 percent over a year ago in a region of 2961 thousand hectares. A production of 10.074 million bales of cotton was recorded this year which were 27.8% less than previous year (Economic Survey of Pakistan, 2015-16).

Being the main cash crop of Pakistan, it is threatened by broad and narrow weeds, and by a number of lepidopteron larvae like spotted bollworms, American bollworms, pink bollworms and Armyworms. These larvae cause direct and great yield loss by feeding on bolls or flowers. In three cotton cultivating provinces of Pakistan like, Punjab, Sindh and Baluchistan, 16 B. thuringiensis cotton varieties developed by public and private institutions have been approved by Punjab seed council between 2010-2012. Out of sixteen cotton varieties, 15 comprises single B. thuringiensis gene Cry1Ac, while the rest one and the only is B. thuringiensis Hybrid which is developed by the fusion of Cry1Ac & Cry1Ab genes (Tabashink et al., 2013).

Pest and virus attack on cotton has significantly mired cotton production but adoption of single B. thuringiensis gene cotton over the area of 85% in Pakistan cannot be neglected and considered among the one of the main reason of stagnancy in cotton production because incidence of pink bollworms and other lepidoptearns insect pests have been reported during 2013-14 (Khuhro et al., 2015). Pink bollworms attacks on fruiting bodies of cotton ranges from 25-30% and has become serious threat. Recently, in Sindh, pink bollworm (Pectinophora gossypiella) has become a real threat to conventional and B. thuringiensis cotton varieties (Kanher et al., 2015). Considering above scenario, one can imagine the awful future with increasing incidence of insect resistance against B. thuringiensis toxins. Instead of killing, the lower expression of B. thuringiensis toxins is helping towards development of resistance of lepidopteron larvae and major threat of using cry toxins in transgenic plant is the appearance of insect resistance (Jin et al., 2015). Documentation of five different insect pest species resistance has been reported (Sparks et al., 2015). Pyramiding strategy that comprises the production of two or more toxins against same pest seems to be the most reliable in order to delay the evolution of pest resistance to transgenic crops (Fukuoka et al., 2015). So, it is the need of the time that development of genetically modified cotton cultivars with stacked B. thuringiensis genes with elevated expression against insect pest, other than cry1Ac should developed. The aims of current innovation is to transform multiple stacked genes in cotton with enhanced constitutive expression to combat with insect pest and development of insect/pest resistance

Methods of Removing Weeds from Crop Field

The sunlight and nutrients of plants are being competed between crops and unwanted weeds. This competency leads to often substantial yield loss. Tandem techniques of soil tilling or herbicide application are adopted by farmers to control weeds at their farms conventionally. Burdensome and needs intensive labor, time and money. Herbicide, on the other hand have not any distinguished between plants and weeds plants. Selective herbicides are the only options as for as conservative agriculture system is concerned. Such herbicides do not damage the crops but at the same time are not equally effective against different types of weeds plants. This problem can be solved by the farmers by using herbicide resistant crops, and by this they can remove all types of weeds with single and swift application of non-selective herbicides. It is time, labor and cost saving needing less spraying, labor and less traffic on the filed with lower operating charges.

Glufosinate is broad spectrum non-selective post emergent herbicide. It belongs to organic phosphorous family of herbicides. It inhibits glutamine synthesis (GS) and nitrogen assimilation ability of GS. Inhibition of GS leads to accumulation of ammonia and finally indirect inhibition of photosynthesis and then weeds plant death. Glufosinate-ammonium ensures a high degree of crop safety, as it only affects the parts of the plant where it is applied. Its unique mode of action makes it ideal to be used in rotation with other herbicides to mitigate weed resistance There are several means for the modification of crops to be tolerant to glufosinate. One approach is to genetically engineer crop plant with Re-PAT, a marine bacterium (Rhodococcus sp. strain YM12) gene that yield resistant against glufosinate. The implementation of glufosinate-based crop production system is one of the most significant revolutions in the history of agriculture.

### Methods of Controlling Insect Pest Infestation in Plants

The proteninaceous parasporal crystals (Cry proteins) which are very well known for their toxin production against a variety of insects including Lepidopteron, Coleopteran, and Dipteran insect larvae are sourced out from gram positive soil bacterium called Bacillus thuringiensis. During sporulation phase of B. thuringiensis crystal proteins are produced and are primarily toxic to certain crop infesting species of insects. The crystal toxic proteins from various compositions comprising B. thuringiensis strains with insecticidal activity have been used commercially being as environmentally-acceptable topical insecticide due to their target specific toxicity to insects and non-toxicity to plants and other microbial consortia. Insect larvae ingest these δ-endotoxin crystals which after solubilization in the mid-gut of the insect release pro-toxin form δ-endotoxin that is processed subsequently to an active toxin by mid-gut protease. Through receptor proteins these activated toxins recognize and bind to the brush-border membrane of the insect mid-gut epithelium and cause the death of insects. Molecular biologists have recognized and isolated various δ-endotoxin genes and determined their DNA sequences just due to Molecular genetic techniques. These genes were further truncated for effective use in higher crop plants and have been approved for commercial use.

### SUMMARY OF THE INVENTION

An aspect of the invention is to develop recombinant polynucleotide sequences of herbicide tolerant Re-PAT (Rhodococcus sp. strain YM12, marine bacterium) gene (SEQ ID NO: 11), two B. thuringiensis insecticidal δ-endotoxin Cry10A (SEQ ID NO: 12) and Cry11a12 (SEQ ID NO: 13) insecticidal gene, to transform into mono and/or dicot plants particularly cotton plants.

Another aspect of the invention is to develop a recombinant polynucleotide sequences of herbicide tolerant Re-PAT (Rhodococcus sp. strain YM12, marine bacterium) gene, two B. thuringiensis insecticidal δ-endotoxin Cry10A and Cry11a12 insecticidal protein gene in stacked triple-gene, with enhanced expression in the transgenic mono and/or dicot plants specifically cotton plants.

Another aspect of the invention is to develop enhanced assembled expression of Re-PAT, Cry10A and Cry la12 genes to make transgenic triple gene plant particularly cotton plant, more tolerant and effective in controlling to broad and narrow leave range of weeds and lepidopteron insect and pest family compared to single gene transgenic plants or cotton plants.

One another aspect of the invention is to develop an identification of recombinant polynucleotide sequences identified as SEQ ID NOS: 1-10 that are useful as primer sequences for the detection of the respective recombinant polynucleotide sequences.

In addition to above, another aspect of the present invention is to develop a recombinant polynucleotide sequence identified as SEQ ID NO: 14 to offers a superior strategy for demolishing of insect resistance by enhanced collective expression of herbicidal gene Re-PAT, and insecticidal proteins encoded by Re-PAT, Cry10A and Cry1a12 genes within the single T-DNA even all three including two insecticidal genes have not significant homology with each other.

Another aspect of the invention, methods or assays for detecting the presence of the transgene insertion region identified as SEQ ID NO: 19 in genome of transgenic plant specifically in cotton plant.

Further adding aspects includes a method for the enhanced expression of Re-PAT, Cry10A and Cry1a12 proteins conferring resistance against insects and pests by expressing them constitutively in plants, cotton plant.

In an exemplary embodiment of the present invention, a profusion of cassettes having a recombinant polynucleotide sequences encompasses a triple gene (SEQ ID NO: 14) with a 5′ end attached through a promoter joined to an un-translated enhancer (intron) sequence and a 3′ end attached to a NOS terminator sequence, for encoding the polynucleotide sequences, wherein the triple gene comprises one Re-PAT herbicidal gene (SEQ ID NO: 11) and two B. thuringiensis δ-endotoxin Cry10A (SEQ ID NO: 12) and Cry11a12 (SEQ ID NO: 13) genes that are encoded to form a herbicidal and insecticidal toxin proteins in a transgenic plant, resulting in decreased resistance development against insecticidal toxins proteins and increased efficacy against the insect mortality.

A first cassette, a second cassette, and a third cassette can be located within a T-DNA region of a vector flanked by a left and a right border sequence.

The first cassette coding the Re-PAT gene having the (SEQ ID NO: 11) can be operably linked to the promoter, the un-translated enhancer sequence at a 5′ end and the NOS terminator sequence tagged to a 3′ end of the Re-PAT gene. The second cassette coding insecticidal Cry10A gene having (SEQ ID NO: 12) can be operably linked to the promoter, the un-translated enhancer sequence at a 5′ end and the NOS terminator sequence tagged to a 3′ end of the gene Cry10A. The third cassette coding the insecticidal Cry11a12 gene having (SEQ ID NO: 13) can be operably linked to the promoter, the un-translated enhancer sequence at a 5′ end and the NOS terminator sequence tagged to a 3′ end of the Cry11a12 gene.

The 5′ end of each gene, the Re-PAT, the Cry10A and the Cry11a12 gene in the transgenic plants can be attached with the un-translated enhancer sequence (intron) comprising 28 nucleotides of SEQ ID NO: 14 starting from the 685^{th} nucleotide to 712^{th} nucleotide.

The promoter can be the Cauliflower mosaic virus (CaMV35S).

The profusion cassettes can have (SEQ ID: 14) present in the transgenic plant, seed, cell or part of the transgenic plant.

The transgenic plant can be a monocot plant selected from the group consisting of sugarcane, wheat and maize. The transgenic plant can be a dicot plant selected from the group consisting of cotton, potato and tomato.

The profusion cassettes can be located at a SEQ ID NO: 19 having a forward primer of SEQ ID NO: 20 and a reverse primer of SEQ ID NO: 21 for identification.

In another exemplary embodiment of the present invention, a recombinant DNA molecule comprises a polynucleotide sequence selected from the group consisting of SEQ ID NOS: 1-14, SEQ ID NO: 19, SEQ ID NO: 20, SEQ ID NO: 21, and complements thereof.

The DNA molecule can comprise SEQ ID NOS: 1-14, SEQ ID NO: 19, SEQ ID NO: 20, SEQ ID NO: 21, and complements thereof, in the transgenic cotton plant, plant cell, seed or plant part.

The DNA molecule can comprise SEQ ID NO: 19 in the transgenic cotton plant, plant cell, seed or plant part. The DNA molecule can comprise SEQ ID NOS: 1-13 in the transgenic cotton plant, plant cell, seed or plant part. The DNA molecule can comprise SEQ ID NOS: 11-13 in the transgenic cotton plant, plant cell, seed or plant part.

In another exemplary embodiment of the present invention, a transgenic cotton plant, seed, cells or plant part can comprise a recombinant DNA molecule comprising a polynucleotide sequence selected from the group consisting of SEQ ID NOS: 1-14, SEQ ID NO: 19, SEQ ID NO: 20, SEQ ID NO: 21, and complements thereof, wherein an amplicon comprises the DNA molecule having the sequence of SEQ ID NOS: 1-10.

In another exemplary embodiment of the present invention, a transgenic cotton plant, seed, cells or plant part can comprise a recombinant DNA molecule comprising a polynucleotide sequence selected from the group consisting of SEQ ID NOS: 1-14, SEQ ID NO: 19, SEQ ID NO: 20, SEQ ID NO: 21, and complements thereof, wherein an amplicon comprises the DNA molecule having the sequence of SEQ ID NO: 14.

### BRIEF DESCRIPTION OF THE SEQUENCES

The following nucleotide sequences make part of current narration and are given to further corroborate certain characteristic of the present invention. Reference regarding these sequences may enhance the vision and scope of the present invention and specific embodiments described herein.

SEQ. ID NOS: 1-2 forward and reverse primers to amplify Re-PAT gene.

SEQ. ID NOS: 3-4 forward and reverse primers to amplify Cry10A gene.

SEQ. ID NOS: 5-6 Forward and reverse primers to amplify Cry11a12 gene.

SEQ. ID NOS: 7-8 Forward and reverse primers to amplify UTR and Re-PAT gene.

SEQ. ID NOS: 9-10 Forward and reverse primers to amplify Cry10A, UTR, terminator and Cry11a12 gene.

SEQ. ID NO: 11 Polynucleotide sequence of Re-PAT gene.

SEQ. ID NO: 12 Polynucleotide sequence of Cry10A gene.

SEQ. ID NO: 13 Polynucleotide sequence of Cry11a12 gene.

SEQ. ID NO: 14 Polynucleotide sequence of T-DNA having all three genes cassette.

SEQ. ID NO: 15 Polypeptide sequence of Re-PAT precursor protein.

SEQ. ID NO: 16 Polypeptide sequence of Cry10A precursor protein.

SEQ. ID NO: 17 Polypeptide sequence of Cry11a12 precursor protein.

SEQ. ID NO: 18 Polypeptide sequences of CaMV 35s promotor & enhancer.

SEQ ID NO: 19 Polynucleotide sequence of synthetic recombinant construct and Gossypium hirsutum genome junction event sequence.

SEQ ID NO: 20 Polynucleotide sequence of primer from synthetic inserted DNA sequence for event detection.

SEQ ID NO: 21 Polynucleotide sequence of primer from Gossypium hirstum genome for event detection.

### DETAILED DESCRIPTION OF THE INVENTION

The following definitions and methods are provided to better define the present invention and to guide those of ordinary skill in the art in the practice of the present invention. Unless otherwise noted, terms are to be understood according to conventional usage by those of ordinary skill in the relevant art.

As used herein, the term “cotton” means Gossypium hirsutum and includes all plant varieties that can be bred with cotton, including wild cotton species.

As used herein, the term “comprising” means “including but not limited to”.

A transgenic “event” is produced by transformation of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the genomic/transgene DNA. Even after repeated back-crossing to a recurrent parent, the inserted transgene DNA and flanking genomic DNA (genomic/transgene DNA) from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

To facilitate an understanding of the principles and features of the various embodiments of the invention, various illustrative embodiments are explained below. Although exemplary embodiments of the invention are explained in detail, it is to be understood that other embodiments are contemplated. The further scope of this present invention will be further highlighted by following content relevant to the present invention, in aggregation with related sequence listing.

Identification and Isolation of Insecticidal Toxin Genes

The technique articulated in this innovation is expected to be utilized to accomplish enhanced expression of Re-PAT, and B. thuringiensis endotoxins Cry10A and Cry11a12 as given below. How to identify new insecticidal sequences have been described well by Donovan et al. 1992, which comprised with following steps: Isolation of tentative insecticidal toxins, amino acid sequencing, back translation, designing of oligonucleotide probe followed by identification and cloning of toxins gene by hybridization. Perlak et al., (1991) used two approaches to increase the toxin levels in genetically modified plants. First those DNA sequences which inhibits excellent plant expression both at translational and mRNA level were selectively removed through side directed mutagenesis to partially modified the gene called (PM) without changing the amino acid sequences. The 2^{nd} one was called fully modified synthetic gene FM. Comparing with wild type, PM sequences had a 10-fold higher expression for insect control while FM sequences had 100-fold higher expression. We used the fully modified gene with GC rich contents to produce higher transcription and translation cellular process in cotton plants.

### Designing of Plant Expression Vectors

Construction of plant expression vectors for aiming tissue specific expression of gene comprises specific constitutive expression promoters along with some tissue specific regulator elements like enhancer sequence. Promoters which direct constitutively enhanced expression in plant tissues will be well known to those of skill in the art in light of present discovery. To obtain enhanced constitutive expression, constitutive promoter and enhancer must be attached at 5′ of constitutively expressed gene supplemented with terminator sequence at 3′ of the expressed gene. For example, when Cry10A an insecticidal gene is expressed under Cauliflower Mosaic Virus 35S promoter, it will express in all tissues. Alternatively, other sources of constitutive promoter may be used for targeting expression of Re-PAT, Cry10A, and Cry11a12 genes.

In the Pakistani agriculture, there exists a stipulation to raise a cotton plant shows characters with multiple and accumulative resistance to reduce yield loss due to variety of lepidopteron insect pests. In the present invention the triple gene cotton plant would reduce the need to apply different chemical and pesticides that might be harmful to other beneficial insects and most importantly to environment. Further, triple gene with different mode of action would help in delaying Lepidopteron insect pest's resistance to B. thuringiensis δ-endotoxin Cry10A and Cry11a12 insecticidal protein genes, which is prevalent problem in Pakistani agriculture with single gene cotton.

The present innovation relates to herbicide and Lepidopteron insect pest resistant Next generation triple gene transgenic cotton under single T-DNA. It likewise identifies with strategies for recognizing plant materials resulting there from. In the frame work of this application genetically modified herbicide and Lepidopteron pest resistant cotton denotes the innovative triple gene transgenic cotton described herein.

Further adding, the invention also includes a method for the enhanced expression of Re-PAT, and two B. thuringiensis δ-endotoxin Cry10A and Cry11a12 insecticidal protein genes conferring resistance against insect pest by expressing them constitutively with un-translated enhancer sequences in the cotton plants.

In first construct cassette, the present invention provides a G. hirsutum codon optimized purified DNA construct comprising synthetic Re-PAT synthase protein-encoding region constitutively or localized to a plant cell nuclear genome. In some embodiments, the Re-PAT gene comprises the sequence of SEQ ID NO: 11.

The second cassette of present invention provides a G. hirsutum codon optimized purified DNA construct cassette comprising synthetic Cry10A protein encoding region localized to constitutive or localized to a plant cell nuclear genome and possibly linked to a region encoding constitutive directed sequence which is one means of enabling expression of Cry10A protein in whole cotton plant. The Cry10A gene comprises the sequence of SEQ ID NO: 12

In the third cassette, the current invention provides cotton codon relevant optimized DNA construct comprising B. thuringiensis insecticidal δ-endotoxin Cry11a12 insecticidal protein gene constitutively localized or localized to a plant cell nuclear genome and is possibly linked to a region of sequence directed constitutively which is one means of enabling localization of Cry11a12 protein to all parts of cotton plant. The Cry11a12 gene comprises the sequence of SEQ ID NO: 13.

### Nucleic Acid Composition

In one exemplary embodiment, the triple gene cotton exhibits a novel genotype comprising the three cassettes of genes and a selectable marker gene.

In the first cassette an appropriate constitutive promoter is attached to a gene that encodes for Re-PAT protein which confers resistance to transgenic cotton plant tolerant to broad and narrow range of weeds.

In the second cassette an appropriate constitutive promoter is attached to a gene that encodes for Cry10A protein which confers to resistance transgenic cotton plant tolerant to lepidopteron pest range of insects.

In the third cassette, Cry11a12 gene is tagged at N-terminal with same 28 bp enhancer sequence for tagged constitutive expression of B. thuringiensis δ-endotoxin Cry11a12 insecticidal protein gene in transgenic cotton plant.

A 28 nucleotides long un-translated enhancer sequence is the identical to the sequence of 28 nucleotide of SEQ ID NO: 14 starting from the 685^{th} nucleotide to the 712^{th} nucleotide.

The already incorporated marker gene into plant expression vector, which when expressed can be used as selection marker. In one embodiment of the present invention selectable marker gene is kanamycin or hygromycin.

All the transgenes (Re-PAT, and B. thuringiensis δ-endotoxin Cry10A and Cry11a12 insecticidal protein genes), at their C terminals are linked separately to polyadenylation signals from Agrobacterium tumefaciens nopaline synthase (NOS) terminator.

The all three cassettes might be inserted into plant on the same or different plasmids.

The first, second, and third cassettes exist on the same plasmid and are introduced into cotton genome by using Agrobacterium-mediated transformation method; they may be present on different or same T-DNA regions:
In one embodiment, all three cassettes are present on the same T-DNA region.
In a second embodiment, the first and second cassettes are present at same T-DNA region.
In a third embodiment, the second and third cassettes are present on same T-DNA region.
In a fourth embodiment, the first and third cassettes are present on same T-DNA region.

### Single T-DNA Triple Gene Creation Method

One herbicide gene named Re-Pat (SEQ ID: 11) and two insecticidal genes Cry10A (SEQ ID: 12), Cry11a12 (SEQ ID: 13) went under codon optimization in a way to make them cotton genome specific. Individual super constitutive promoter with dicot specific untranslated enhancer regions attached with each gene at its 5′ end along with its terminator sequence at 3′ end to constitute a complete Single T-DNA. So, overall it was constituted 7578 bp single T-DNA construct (SEQ ID: 14).

### Transformation in Cotton

A very well-known cotton transformation and regeneration procedure present are usually Agrobacterium tumefacines based mediated transformation of foreign DNA into cotton genome and regeneration of cotton plant parts mostly immature embryos into fully productive genetically modified cotton plants. Mostly dicotyledonous, but some time monocotyledonous plants are transformed by using Agrobacterium-mediated transformation, but it is more effective against dicotyledonous like cotton plants. The cloning of DNA of interest is done in binary expression vector between left and right T-border consensus sequences, called T-DNA region. The binary vector harboring DNA of interest is transmitted to Agrobacterium cell via electroporation method. The electroporated transmitted binary expression vector is then co-cultivated with cotton embryos.

The binary vector comprising the DNA of interest under T-DNA region is then integrated into cotton plant genome. The featured gene cassette and selectable marker gene cassette may be present on the same T-DNA regions in the same vector or vice versa.

In one embodiment of the present innovation, the gene cassettes are present on the same T-DNA region.

After transformation, the next step is the selection regeneration of putative transgenic plants via antibiotic drug application to appropriate marker gene (kanamycin or hygromycin) and progeny retaining the foreign DNA. The composition of suitable regeneration medium is well known to any skilled man.

The transgenic plants achieved thus, as described in the present invention, have herbicidal or insecticidal effects. These plants showed tolerance to-non-selective herbicide sprays and are resistant to Lepidopteron, Coleopteran, and Dipteran insect larvae which may attack on it. Subsequently, self-defense mechanism is shown by the transgenic cotton plants of the present invention against invasion by lepidopteron species comprising Heliothis sp., Helicoverpa sp., Pictinophora sp. and Spodoptera sp. A fewer insecticide sprays are needed for cultivation of invented transgenic cotton plants in comparison to wild-type plants of the same cultivars and minimal loss of yield through insect pest has been observed.

The current innovation is not limited to the aforementioned transgenic cotton plants only but is endorsed comprehensively to take account of any plant material gained from them including seed if at least one of the current invention polynucleotide is contained by them.

The present invention keeps plants which are obtained from breeding crosses with the current transgenic cotton plants or resultant there from by orthodox breeding or any other procedure.

The plant material attained from the transgenic plant that may contain additional, changed or fewer polynucleotide sequences matched with genetically modified cotton plants is also covered under this present invention. For example, if someone desires to generate a new event by with the transgenic cotton plant or display other phenotypic features, such as a third insect resistance gene a procedure well-known as gene stacking.

The current innovation also provides methods to obtain higher constitutively targeted expression of herbicide tolerant Re-PAT, insecticidal genes Cry10A and Cy1la12 in dicotyledonous transgenic plants, without disturbing the normal phenotype and agronomic characteristics of the transgenic plants.

The present invention also allows getting insecticidal toxins at levels up-to 27 times higher than that shown by existing procedures.

The present invention enables transgenic plants expressing Re-PAT, Cry10A, and Cry11a12 gene to be used as an alternative to plants expressing first generation single genes toxins. These next generation toxins with their combined effect will be used both for control as well as resistance management of significant lepidopteron insects range as mentioned above. It is also predicted that two insecticidal toxins having different mode of action in the insect mid gut will enhance the effectiveness against target insect pest and will decrease the possibility of developed resistance against these toxin proteins. The higher constitutive expression will further reduce the chances of insect resistance.

The method of expressing triple gene Re-PAT, two B. thuringiensis insecticidal δ-endotoxin Cry10A and Cry11a12 insecticidal protein gene assembled constitutively in cotton plants includes the following steps:

i) Designing and constructing a polynucleotide consisting of a suitable promoter joined to an un-translated enhancer sequence which is further tagged to DNA sequence encoding herbicidal and insecticidal proteins Re-PAT and two B. thuringiensis insecticidal δ-endotoxin Cry10A and Cry11a12 insecticidal protein gene which are further tagged with suitable terminator sequence.

ii) The genes thus tagged with constitutive promoters to express with assembled proteins and consequently increased combined toxin
iii) Transforming the cotton plants with DNA construct of step (i) so that transgenic plant expresses combined proteins in all tissue of cotton plants.

Any cultivar of dicotyledonous plant including fiber, fruit, legume tuber and any variety of species of monocotyledonous plant is covered by the present invention.

In preferred incarnations, the dicot is a cotton, tomato and potato plant or cell, while maize, rice wheat and sugarcane are preferred embodiments of monocot plant.

### Laboratory Insect Bioassays of Transgenic Plant Events

The identification of transgenic cotton plant expressing high level of two B. thuringiensis insecticidal δ-endotoxin Cry10A and Cry11a1 protein of interest and herbicide tolerance, screening is essential of the antibiotic resistant transgenic regenerated plants (To generation) for insecticidal activity and/or expression of interest. Numerous methods well known by those skilled in the art of may help in completion of this task, including but not limited to (1) taking leaf samples from the transgenic T₀ plants and directly going for assay the leaf for activity against susceptible insects in comparison with tissue obtained from a non-transgenic, negative control cotton plant. For example, T₀ cotton plants expressing Re-PAT, two B. thuringiensis insecticidal δ-endotoxin Cry10A and Cry11a12 protein gene can be identified by assaying leaf tissue obtained from such plants for activity against lepidopteron species. (2) Analysis of extracted protein samples by Enzyme Linked Immuno Sorbent Assay (ELISA) specific for the gene of interest (Re-PAT, Cry10A and Cry11a12): or (3) reverse transcriptase PCR™ to identify events of the expression of genes of interest.

### Method of Expressing Herbicide Re-Pat, and Two Insecticidal δ-Endotoxin Cry10a and Cry11a12 (Bacillus thuringiensis) Gene Proteins in Progeny Plant

The author of this invention further anticipates that the method revealed in this invention comprises a method of generating a transgenic progeny plant. The method of generating such progeny includes: the process of expressing Re-PAT, δ-endotoxin Cry10A and Cry11a12 herbicidal and insecticidal toxin in a plant disclosed herein includes steps of: (i) Designing and constructing a polynucleotide consisting of suitable constitutive promoter operably joined to a un-translated enhancer sequence which is further tagged to DNA sequence encoding Re-PAT, δ-endotoxin Cry10A and Cry11a12 herbicidal & insecticidal proteins which is further linked at 3′ end to a suitable terminator sequence. Thus, these genes attached with subsequent constitutive promoters and enhancers sequences will produce combined toxin proteins. (ii) Procuring a second plant: and (iii) crossing the first and second plants to get crossed transgenic progeny plant that has innate the nucleic acid segments from the first plant. The current innovation precisely includes the progeny plant or seed from any of the transgenic plants, dicot or monocot containing the whole or partial polynucleotide sequence (SEQ ID NO: 14)

### Cloning and Vector Construction

Agrobacterium-mediated transformation vector construction was typically based on employing the restriction digestion and ligation techniques for cloning in sub vectors. The plasmid vector was comprised of the following cassettes: (i) a gene cassette containing Cauliflower mosaic virus (CaMV35S) sequence, a un-translated enhancer sequence, a sequence encoding cotton-optimized synthetic Re-PAT gene conferring herbicidal resistance and a NOS polyadenylation sequence; (ii) second cassette loaded with Cauliflower mosaic virus (CaMV35S), un-translated enhancer sequence, a sequence encoding the cotton-optimized Cry10A gene and a NOS polyadenylation terminator sequence; (iii) the third gene cassette consist of Cauliflower mosaic virus (CaMV35S) promoter, dicot specific expression enhancer sequence, a sequence encoding cotton-optimized Cry11a12 gene and a NOS polyadenlation terminator sequence (iv) and the already incorporated sequence i.e. marker gene that encoding protein conferring resistance to hygromycin or kanamycin and a NOS polyadenylation sequence.

These gene cassettes were cloned within T-DNA region of vector p4bT3 flanked by left and right border sequences and by employing standard Agrobacterium electroporation transformation technique, the above gene constructs were transformed into Agrobacterium tumefaciens strain LB 4404 and the transformed cell culture were selected through kanamycin.

### Cotton Plant Transformation

Regeneration of transgenic cotton plants was done by using standard agrobacterium-mediated transformation method by using germinating embryos of G. hirsutum cv. FBS-286 and Eagle-2 as optimized by Ali et al., (2016).

Sterilization of FBS-286 and Eagle-2 delinted seeds was done for 60 seconds by using 10% SDS and 5% Mercuric chloride with enough water covering seeds and continuous shaking of flask. Subsequent washing of seeds was done until no foam was seen. Finally, the washed seeds were further soaked with 10 ml sterilized distilled water. The flask was covered with dark cloth and seeds were allowed to germinate at 30° C. for continuous 36 hours. A 10 ml culture of agrobacterium comprising of p4bT3 was grown under suitable antibiotic selections in YEP broth medium. The pellet was dissolved in autoclaved simple MS broth medium after centrifuging it for 10 minutes at 4° C. By removing seed coat and cotyledons tissue germinating seeds were taken out manually. A minor cut towards shoot-apex was given to each isolated embryo with sterilized blade and then put into diluted agrobacterium culture supplemented with acetosyringone (Sigma-Aldrich™) and were allowed to co-cultivate for 1 hour at shaker set at 30° C.

Embryos treated with agrobacterium were blotted on autoclaved filter paper for removing excess bacteria. The embryos were then transformed on petri plates comprising kanamycin and MS medium (MS salt, 4.43 g/L, B5 vitamin, 2 mg/L NAA, 0.1 mg/L kinetin, 30 g/L sucrose, 3.5 g/L Phytogel and 200 mg/ml cefotaxime sodium-salt, pH 5.7). The plates were incubated at 28° C. in the light for 4-6 days after wrapping with paraffin film. The embryos grew in size and turned green. At fifth day, healthy and suspected kanamycin resistant embryos from plates were shifted to 25×200 mm test tubes containing MS media (Same composition as above) supplemented with proper antibiotic again and kept under 14 hours of light and 10 hours at dark conditions for three to four months until healthy shoots were developed. During this period after three weeks shoots were transferred into fresh MS selection free media for rapid growth.

Fully developed and healthy plants were then further transferred to MS medium supplemented with rooting hormones and without antibiotic selection. The plants with healthy roots were then given name putative transgenic plants were shifted to small pots having soil, peat and bhall in certain ratios. The plants were then acclimatized.

### Identification and Selection of Transgenes

The Genomic DNA was extracted from putative transgenic cotton plants and tested through standard polymerase chain reaction techniques by employing gene specific primer sequences (SEQ ID NOS: 1-6) for the existence of transgenes (Re-PAT, δ-endotoxin Cry10A and Cry11a12 insecticidal protein gene).

The positive plant events were identified and went go through screening process of Laboratory insect bioassay for their insecticidal activity lepidopteron species comprising Heliothis sp., Helicoverpa sp., Pictinophora sp. and Spodoptera sp. and herbicidal spray in a controlled containment.

### Antibodies Production

Two replications of three male albino rabbits approximately weighing 1.5 kg were intravenously injected at multiple sites separately with purified antigen of Re-PAT, Cry10A and Cry11a12. The Rabbits were fed properly and were injected with respective proteins after further fifteen days. Taking 5 ml blood of each rabbit antibody titer was checked by ELISA. Whole blood was isolated after two months by cardiac puncturing. Serum was stored at −20° C. after isolating with standard procedures. Pre-immune control serum was obtained from rabbits before immunization.

### Antibodies Purification

Rabbit's monoclonal anti-Re-PAT, anti-Cry10A, anti-Cry11a12 antibodies were purified on protein affinity resin. Antibody purified were dialyzed against PBS, dispensed in aliquots and stored frozen at −20° C. ELISA titer was again carried out to check activity of each purified antibody.

Antibodies Characterization

Protein Extraction

Plant material of 200 mg (leaves, root, and stem) from transgenic as well as non-transgenic cotton plants ground in liquid nitrogen in pre-chilled sterile mortar and pestle. Proper dry ground powder was transferred to 1.5 ml micro tube and was supplemented with 300 μl protein extraction buffer (0.5M EDTA, 0.5M NaCl, 20 mM Tris-HCL pH7.5, 20 mM NH4Cl, 0.5m PMSF, 10 mM DTT and 0.5M Glycerol). Samples were incubated for one hour-overnight at 4° C. after homogenization by vortexing, and went to centrifugation for 15 minutes at 4° C. at maximum speed. Supernatant was taken, and Bradford reagent extracted protein was quantified on spectrophotometer. For further analysis samples were diluted with 1:10.

Enzyme Linked Immunosorbent Assay

The Re-PAT, Cry10A and Cry11a12 expressed proteins (SEQ ID: 15-17) were detected by indirect ELISA. Plant protein samples were denatured in boiling water for 10 minutes and were mixed with 50 mM carbonate buffer (pH, 9.5) and dispensed into 96 well micro titer-plates accordingly and went for incubation at 37° C. three hours to overnight. Tris buffer saline and Tween 20 were used for rinsing unbound antigen. The BSA/TBS blocking buffer (5%) was employed for blocking of unbound non-specific sites and endorsed to bind with anti-Re-PAT, Cry10A and Cry11a12 antibodies respectively.

The bound antibodies were detected by goat anti-rabbit IgG after standard washing using BCIP/NBT substrate. 1N HCL was used to stop the ELISA reaction. Absorbance was taken at 430 nm spectrum, using negative control as blank. Using standard between optical densities of different concentration of standard a graph was plotted. The respective concentrations of Re-PAT, Cry10A and Cry11a12 were determined by placing their respective OD values on standard graph. The protein was quantified by using the following formula.

$$Transgenic protein ( μ \text{/} g leaf tissue ) = Conc . on graph \times \frac{[ 500 \times mg of tissue taken ]}{1000} \times dilution factor$$

Immuno Dot Blot

For quick screening of samples having Re-PAT, Cry10A and Cry11a12 expressed proteins of transgenic cotton plants an Immuno Dot Blot analysis was carried out. A triple gene purified denatured protein samples of transgenic and non-transgenic plants were applied onto nitrocellulose membrane. After drying unbound parts of the membrane were blocked with 5% blocking buffer (BSA/TBS). A primary antibody (anti Re-PAT, Cry10A and Cry11a12 rabbits 1:10000) was added after washing with thrice with 1×PBS and incubated at 37° C. for one hour. The blot was incubated with secondary IgG (anti-IgG Rabbit mouse AP-conjugated) after given three washing with 1×PBS. After one hour, blot was washed again three times with lx PBS and BCIP/NBT substrate was added and incubated at 37° C. for 30 minutes for detection of transgenic proteins.

Genomic DNA Extraction

The total genomic DNA was isolated from leaves of transgenic cotton plants by using CTAB method. A 300 mg sample from leaves was plucked and put immediately into liquid nitrogen container for grinding. Each sample went to fine grinding in pre-chilled Mortar Pestle by using liquid nitrogen. A fresh Eppendorf was loaded with fine ground powder and mixed through with added pre-heated DNA extraction buffer (2% CTAB, 1% Mercapto-ethanol, 2 mM NaCl, 200 mM EDTA, RNase A and 100 mM Tris-HCl).

After incubation at 65° C. for one hour added one volume of phenol (pH: 8), vortexed, spun for 10 minutes at maximum speed. Supernatant was further treated with equal volume of Chloroform: Isoamyalchol (24:1) and spun. After having supernatant again 0.7 volume of isopropanol was added and kept at −20° C. for overnight. After spinning next day pellet was washed twice with 70% chilled ethanol, and re-suspended in 50 μl sterile water after air drying. DNA was quantified on 0.8% agarose gel.

Similarly, Genomic DNA from leaf tissues of Kalgin-4 positive transgenic plant was isolated by using above mentioned protocol to find the event/location junction between transgenic/Gossypium hirstum genome. Gel was run to quantify the Genomic DNA.

Polymerase Chain Reaction (PCR)

By using gene specific primers (SEQ ID NOS: 1-6) of individual Re-PAT, Cry10A and Cry11a12 genes and cassettes (SEQ ID NOS: 7-10) PCRs were carried out from isolated genomic. A reaction volume of 25 μl was comprised with 150 ng DNA template, both gene specific primers, 20 picomole each dNTPs mix 3 mM lx Taq buffer, 2.5 units of Taq Polymerase (Invitrogen). Reaction was carried out in Applied Biosciences Thermo cycler with the following conditions: 95° C. for 5 min, (95° C. 35 sec, 57° C. 45 Sec, 72° C. 160 sec)×35 cycles and final extension at 72° C. for 10 minutes and was hold at 20° C. for 5 minutes.

To find the transgenic/cotton genome junction a forward primer SEQ ID NO: 20 was designed from the synthetic sequence of insert at 3′end of the recombinant construct. PCR reaction was made by using the following PCR conditions: 95° C. for 5 min, (95° C. 35 sec, 57° C. 45 Sec, 72° C. 120 sec)×35 cycles and final extension at 72° C. for 10 minutes and was hold at 20° C. for 5 minutes.

Agarose Gel Electrophoresis

Agarose gel stained with ethidium bromide (0.5 μg/ml) was used for running of PCR amplified gene fragments in 1% TAE buffer. PCR was mixed with 30 loading dye (bromo-phenol). Electrophoresis was carried out at 100V for 30 minutes in gel electrophoresis apparatus (Bio-Rad) and was observed under UV light in Gel Documentation apparatus (VWR, UK).

A separate gel was run for transgenic/genome junction PCR. PCR product was purified by using Gene JET Gel Extraction kit them scientific (K0701). Purified product get sequenced from Macrogen Sequencing Service Korea.

Transgenic/Genome Junction Polymerase Chain Reaction (PCR)

After getting Sequence results of PCR product a primer from the genome of the cotton was designed (SEQ ID NO: 21). Then by using these both primers a PCR reaction was gain run by using the following conditions: PCR reaction was made by using the following PCR conditions: 95° C. for 5 min, (95° C. 35 sec, 56° C. 45 Sec, 72° C. 120 sec)×35 cycles and final extension at 72° C. for 10 minutes and was hold at 20° C. for 5 minutes, and PCR product was run on gel. After again getting it into sequencing a 537 band was achieved.

Field Test and Observation

After confirming the presence of single T-DNA, in transgenic cotton plants subjected to insect bioassay/field test against two main chewing insects Army worm and Pink bollworm. Approx. 100% mortality against larvae of cotton bollworms specifically Army worm and pink bollworm was observed and no further hatching was seen on the back of the cotton plant leaves as compared to single gene constructs. Previously, with each single gene resistance was developed in army worm and maximum mortality rate was on an average 65% due to under and not up to the threshold expression. So, due to synergistic effect of insecticidal genes a 35% more control against larvae was achieved due to the exact binding of toxins in the mid gut of chewing insects especially army and Pink bollworm as disclosed in prior art.

Further, another test related to herbicide was conducted wherein a 1500 ml/acre of herbicide (glufosinate) spray was tolerated by the cotton plants which is 700 ml more than the recommendations. Even though complete weeds destruction was observed at 800 ml during field test.

Table of Sequences

| SEQ | | | |
|---|---|---|---|
| ID | | | |
| NO. | Type | Source | Sequence |
| 1 | DNA | Artificial | ATGTTGATTAGGGATGCTGTTCC |
| | | Sequence | |
| 2 | DNA | Artificial | ATATCAAGCCATCTTCCGAATTT |
| | | Sequence | |
| 3 | DNA | Artificial | ATGCTAGAAATAAGGCTTTGACTGAT |
| | | Sequence | |
| 4 | DNA | Artificial | AATCGTACTCAGCTTCATAAGTAACC |
| | | Sequence | |
| 5 | DNA | Artificial | ATCAGAACAAGAACGAGTATGAGATTT |
| | | Sequence | |
| 6 | DNA | Artificial | CGTTGGTAGCATATCTAATCCTAACTT |
| | | Sequence | |
| 7 | DNA | Artificial | CGCTGGAATTCTAGTAGATGCTG |
| | | Sequence | |
| 8 | DNA | Artificial | GCTCTAGCCCTCTCGATAAGTTC |
| | | Sequence | |
| 9 | DNA | Artificial | TTAAGGCTCTTGTTTATTCATGGACA |
| | | Sequence | |
| 10 | DNA | Artificial | GTTCTGATAAGGGTTCATGGTTTAGT |
| | | Sequence | |
| 11 | DNA | Artificial | ATGTTGATTA GGGATGCTGT TCCTGGAGAT |
| | | Sequence | TTGCCTGGTA |
| | | | TTCTTGAAAT CCATAATGAG GCTATTGCTA |
| | | | ACTCTACTGC |
| | | | TATCTGGGAT GAAACACCTG CTGATTTGGA |
| | | | TGAGAGAAGG |
| | | | AGATGGTTCG ATGATAGGAG AGCTAATGGT |
| | | | TTTCCAGTTC |
| | | | TTGTTGCTGA TGTTGATGGT GTTGTTGCTG |
| | | | GATATGCTTC |
| | | | TTACGGAGTT TGGAGGGCTA AGTCTTCATA |
| | | | CAGACATACT |
| | | | GTTGAAAACT CAGTTTACGT TCATGTTGAT |
| | | | CATCATAGGA |
| | | | GAGGTATTGC TACCGCTTTG ATGACTGAAC |
| | | | TTATCGAGAG |
| | | | GGCTAGAGCT GGTGGTATTC ATGTTATCGT |
| | | | TGCTTCTGTT |
| | | | GAATCAACTA ATGCTACATC AGTTGCTTTG |
| | | | CATGAGAGGT |
| | | | TCGGTTTCAG AATCGTTGCT CACATGCCTG |
| | | | AGGTTGGTAG |
| | | | GAAATTCGGA AGATGGCTTG ATATGACATA |
| | | | TCTTCAATTG |
| | | | ACCCTTTGA |
| 12 | DNA | Artificial | ATGAAACTCA AAAACCAAGA CAAACATCAG |
| | | Sequence | AGCTTCTCCT |
| | | | CAAACGCTAA GGTGGATAAA ATCAGCACAG |
| | | | ACAGCCTCAA |
| | | | AAACGAGACT GATATTGAGC TTCAAAACAT |
| | | | CAATCATGAA |
| | | | GATTGTTTGA AGATGTCAGA ATATGAGAAC |
| | | | GTTGAGCCTT |
| | | | TCGTTTCAGC TTCTACAATT CAAACCGGTA |
| | | | TTGGAATCGC |
| | | | TGGAAAGATT CTTGGTACAC TTGGAGTTCC |
| | | | TTTCGCTGGA |
| | | | CAAGTTGCTT CACTTTACTC TTTTATTTTG GGTGAACTTT |
| | | | GGCCAAAGGG AAAAAATCAA TGGGAAATTT |
| | | | TTATGGAGCA |
| | | | TGTTGAAGAG ATTATCAACC AAAAGATTTC |
| | | | TACTTATGCT |
| | | | AGAAATAAGG CTTTGACTGA TCTTAAAGGT |
| | | | TTGGGAGATG |
| | | | CTCTTGCTGT TTACCATGAT TCATTGGAGT |
| | | | CTTGGGTTGG |
| | | | TAACAGAAAT AACACAAGAG CTAGGTCTGT |
| | | | TGTTAGGTCA |
| | | | CAATATATTG CTCTTGAATT GATGTTCGTT |
| | | | CAAAAATTGC |
| | | | CATCTTTTGC TGTTTCAGGA GAAGAGGTTC |
| | | | CTCTTTTGCC |
| | | | AATCTACGCT CAAGCTGCTA ACCTTCATCT |
| | | | TTTGCTTTTG |
| | | | AGGGATGCTT CAATTTTCGG AAAGGAGTGG |
| | | | GGTCTTTCTT |
| | | | CTTCTGAAAT CTCTACTTTT TACAATAGAC |
| | | | AAGTTGAGAG |
| | | | GGCTGGAGAT TATTCAGATC ATTGCGTTAA |
| | | | ATGGTACTCT |
| | | | ACCGGTCTTA ATAACTTGAG AGGAACTAAC |
| | | | GCTGAATCAT |
| | | | GGGTTAGGTA CAATCAATTC AGAAGGGATA |
| | | | TGACTCTTAT |
| | | | GGTTCTTGAT TTGGTTGCTT TGTTCCCTTC TTATGATACA |
| | | | CAAATGTACC CAATTAAGAC TACAGCTCAA |
| | | | CTTACTAGAG |
| | | | AGGTTTATAC AGATGCTATC GGAACCGTTC |
| | | | ATCCTCATCC |
| | | | ATCATTCACT TCTACCACTT GGTACAATAA |
| | | | CAATGCTCCT |
| | | | TCATTTTCTG CTATTGAGGC TGCTGTTGTT |
| | | | AGGAACCCAC |
| | | | ATCTTTTGGA TTTCTTGGAA CAAGTTACAA |
| | | | TCTATTCTCT |
| | | | TCTTTCTAGG TGGTCAAACA CCCAATACAT |
| | | | GAATATGTGG |
| | | | GGTGGACATA AGTTGGAGTT CAGAACCATT |
| | | | GGTGGAACTT |
| | | | TGAATATCTC TACCCAAGGT TCAACCAACA |
| | | | CTTCTATTAA |
| | | | TCCTGTTACA CTTCCATTCA CCTCAAGAGA |
| | | | TGTTTATAGG |
| | | | ACTGAATCTT TGGCTGGTCT TAACTTGTTT |
| | | | CTTACACAAC |
| | | | CTGTTAATGG TGTTCCTAGA GTTGATTTCC |
| | | | ATTGGAAATT |
| | | | TGTTACTCAT CCTATCGCTT CAGATAACTT |
| | | | CTACTACCCA |
| | | | GGATACGCTG GTATCGGAAC ACAACTTCAA |
| | | | GATTCTGAAA |
| | | | ACGAGTTGCC TCCAGAGGCT ACCGGTCAAC |
| | | | CTAATTATGA |
| | | | ATCATACTCT CATAGGCTTT CACATATTGG |
| | | | ATTGATCTCA |
| | | | GCTTCTCATG TTAAGGCTCT TGTTTATTCA |
| | | | TGGACACATA |
| | | | GGTCTGCTGA TAGGACAAAC ACCATTGAAC |
| | | | CTAATTCTAT |
| | | | TACCCAAATC CCTCTTGTTA AGGCTTTCAA TCTTTCTTCT |
| | | | GGTGCTGCTG TTGTTAGAGG TCCTGGTTTC |
| | | | ACTGGTGGAG |
| | | | ATATTCTTAG AAGGACTAAT ACAGGTACTT |
| | | | TCGGAGATAT |
| | | | TAGGGTTAAC ATCAATCCTC CATTTGCTCA |
| | | | AAGATATAGG |
| | | | GTTAGAATCA GGTACGCTTC AACAACCGAT |
| | | | TTGCAATTCC |
| | | | ATACTTCTAT TAACGGAAAG GCTATCAACC |
| | | | AAGGAAATTT |
| | | | TTCTGCTACT ATGAATAGAG GTGAGGATCT |
| | | | TGATTATAAG |
| | | | ACTTTCAGGA CAGTTGGTTT CACTACACCT TTCTCATTTT |
| | | | TGGATGTTCA ATCTACTTTC ACAATTGGTG |
| | | | CTTGGAACTT |
| | | | TTCATCTGGA AACGAAGTTT ACATCGATAG |
| | | | AATCGAATTT |
| | | | GTTCCTGTGG AGGTTACTTA TGAAGCTGAG |
| | | | TACGATTTCG |
| | | | AAAAGGCTCA AGAGAAAGTT ACCGCTCTTT |
| | | | TTACCTCAAC |
| | | | TAACCCTAGA GGTCTTAAGA CTGATGTTAA |
| | | | AGATTACCAT |
| | | | ATCGATCAAG TTTCTAATCT TGTTGAATCA |
| | | | TTGTCTGATG |
| | | | AGTTTTACCT TGATGAAAAG AGGGAATTGT |
| | | | TTGAAATCGT |
| | | | GAAGTATGCC AAGCAACTCC ACATTGAAAG |
| | | | AAATATGTAA |
| 13 | DNA | Artificial | ATGAACCCTT ATCAGAACAA GAACGAGTAT |
| | | Sequence | GAGATTTTTA |
| | | | ACGCTCCATC CAACGGCTTT AGCAAGTCAA |
| | | | ACAACTACAG |
| | | | CAGGTATCCA CTTGCTAACA AGCCTAATCA |
| | | | ACCATTGAAG |
| | | | AATACCAACT ACAAGGATTG GCTTAATGTT |
| | | | TGCCAAGATA |
| | | | ATCAACAATA TGGTAACAAT GCTGGAAACT |
| | | | TCGCTTCTTC |
| | | | AGAGACAATT GTTGGTGTTT CTGCTGGTAT |
| | | | CATCGTTGTT |
| | | | GGTACAATGC TTGGTGCTTT TGCTGCTCCT |
| | | | GTTTTGGCTG |
| | | | CTGGTATTAT TTCTTTTGGA ACATTGCTTC CTATTTTCTG |
| | | | GCAAGGTTCT GATCCTGCTA ACGTTTGGCA |
| | | | AGATTTGTTG |
| | | | AATATCGGTG GTAGGCCAAT CCAAGAGATT |
| | | | GATAAGAATA |
| | | | TCATTAACGT TTTGACCTCT ATCGTTACAC |
| | | | CTATTAAGAA |
| | | | CCAACTTGAT AAGTACCAAG AATTTTTCGA |
| | | | TAAATGGGAA |
| | | | CCTGCTAGGA CACATGCTAA CGCTAAGGCT |
| | | | GTTCATGATC |
| | | | TTTTCACCAC CTTGGAGCCA ATTATCGATA |
| | | | AGGATTTGGA |
| | | | TATGCTTAAG AACAACGCTT CATACAGAAT |
| | | | CCCTACTCTT |
| | | | CCAGCTTATG CTCAAATTGC TACATGGCAT |
| | | | CTTAATTTGC |
| | | | TTAAGCATGC TGCTACTTAT TACAATATCT |
| | | | GGCTTCAAAA |
| | | | CCAAGGAATC AATCCATCAA CTTTTAATTC |
| | | | ATCTAACTAC |
| | | | TATCAAGGTT ATCTTAAGAG GAAAATTCAA |
| | | | GAGTATACTG |
| | | | ATTACTGCAT TCAAACTTAT AACGCTGGAC |
| | | | TTACCATGAT |
| | | | TAGGACAAAT ACCAACGCTA CTTGGAATAT |
| | | | GTATAATACC |
| | | | TACAGGCTTG AGATGACTTT GACCGTTTTG |
| | | | GATCTTATCG |
| | | | CTATCTTCCC AAATTATGAT CCAGAGAAGT |
| | | | ACCCTATCGG |
| | | | TGTTAAGTCA GAACTTATTA GAGAAGTTTA |
| | | | CACTAATGTT |
| | | | AACTCTGATA CTTTTAGAAC AATCACAGAG |
| | | | TTGGAAAACG |
| | | | GTCTTACAAG AAATCCAACA CTTTTTACCT |
| | | | GGATCAACCA |
| | | | AGGAAGATTC TACACTAGGA ACTCTAGGGA |
| | | | TATTCTTGAT |
| | | | CCTTACGATA TTTTCTCATT CACAGGAAAT |
| | | | CAAATGGCTT |
| | | | TTACTCATAC AAACGATGAT AGGAATATCA |
| | | | TCTGGGGTGC |
| | | | TGTTCATGGA AACATCATCT CACAAGATAC |
| | | | ATCAAAGGTT |
| | | | TTCCCATTTT ACAGGAACAA GCCTATCGAT |
| | | | AAGGTTGAGA |
| | | | TCGTTAGGCA TAGGGAGTAT TCTGATATTA |
| | | | TCTATGAGAT |
| | | | GATCTTCTTT TCAAACTCAT CTGAAGTTTT |
| | | | CAGATATTCA |
| | | | TCTAACTCTA CTATCGAAAA CAATTACAAA |
| | | | AGAACAGATT |
| | | | CTTATATGAT TCCTAAGCAA ACTTGGAAGA |
| | | | ACAAGGAATA |
| | | | CGGACATACT CTTTCTTATA TCAAGACAGA |
| | | | TAACTACATC |
| | | | TTCTCAGTTG TTAGAGAGAG GAGAAGGGTT |
| | | | GCTTTCTCAT |
| | | | GGACTCATAC TTCTGTTGAT TTTCAAAACA |
| | | | CTATTGATTT |
| | | | GGATAACATT ACCCAAATTC ATGCTCTTAA |
| | | | GGCTTTGAAG |
| | | | GTTTCTTCAG ATTCTAAAAT CGTTAAGGGT |
| | | | CCTGGACATA |
| | | | CTGGAGGAGA TTTGGTTATT TTGAAGGATT |
| | | | CTATGGATTT |
| | | | TAGAGTTAGA TTTCTTAAGA ACGTTTCAAG |
| | | | GCAATACCAA |
| | | | GTTAGGATTA GATATGCTAC CAACGCTCCT |
| | | | AAGACAACTG |
| | | | TTTTCTTGAC TGGTATTGAT ACAATCTCAG |
| | | | TTGAACTTCC |
| | | | ATCTACAACC TCTAGGCAAA ATCCAAATGC |
| | | | TACTGATCTT |
| | | | ACTTACGCTG ATTTCGGATA CGTTACCTTT |
| | | | CCAAGAACCG |
| | | | TTCCTAACAA AACTTTCGAG GGTGAAGATA |
| | | | CACTTTTGAT |
| | | | GACATTGTAT GGTACACCTA ACCATTCTTA |
| | | | TAACATCTAC |
| | | | ATTGATAAGA TCGAGTTTAT CCCAATTACT |
| | | | CAATCTGTTT |
| | | | TGGATTATAC AGAAAAGCAG AATATCGAGA |
| | | | AGACCCAGAA |
| | | | GATTGTGAAT GATTTGTTTG TGAACTGA |
| 14 | DNA | Artificial | CCCGGGTCAG CGTGTCCTCT CCAAATGAAA |
| | | Sequence | TGAACTTCCT |
| | | | TATATAGAGG AAGGTCTTGC GAAGGATAGT |
| | | | GGGATTGTGC |
| | | | GTCATCCCTT ACGTCAGTGG AGATATCACA |
| | | | TCAATCCACT |
| | | | TGCTTTGAAG ACGTGGTTGG AACGTCTTCT |
| | | | TTTTCCACGA |
| | | | TGCTCCTCGT GGGTGGGGGT CCATCTTTGG |
| | | | GACCACTGTC |
| | | | GGCAGAGGCA TCTTGAACGA TAGCCTTTCC |
| | | | TTTATCGCAA |
| | | | TGATGGCATT TGTAGGTGCC ACCTTCCTTT |
| | | | TCTACTGTCC |
| | | | TTTTGATGAA GTGACAGATA GCTGGGCAAT |
| | | | GGAATCCGAG |
| | | | GAGGTTTCCC GATATTACCC TTTGTTGAAA |
| | | | AGTCTCAATA |
| | | | GCCCTTTGGT CTTCTGAGAC TGTATCTTTG ATATTCTTGG |
| | | | AGTAGACGAG AGTGTCGTGC TCCACCATGT |
| | | | TATCACATCA |
| | | | ATCCACTTGC TTTGAAGACG TGGTTGGAAC |
| | | | GTCTTCTTTT |
| | | | TCCACGATGC TCCTCGTGGG TGGGGGTCCA |
| | | | TCTTTGGGAC |
| | | | CACTGTCGGC AGAGGCATCT TGAACGATAG |
| | | | CCTTTCCTTT |
| | | | ATCGCAATGA TGGCATTTGT AGGTGCCACC |
| | | | TTCCTTTTCT |
| | | | ACTGTCCTTT TGATGAAGTG ACAGATAGCT |
| | | | GGGCAATGGA |
| | | | ATCCGAGGAG GTTTCCCGAT ATTACCCTTT |
| | | | GTTGAAAAGT |
| | | | CTCAACACGC TGGAATTCTA GTATACTAAA |
| | | | CCATGTTGAT |
| | | | TAGGGATGCT GTTCCTGGAG ATTTGCCTGG |
| | | | TATTCTTGAA |
| | | | ATCCATAATG AGGCTATTGC TAACTCTACT |
| | | | GCTATCTGGG |
| | | | ATGAAACACC TGCTGATTTG GATGAGAGAA |
| | | | GGAGATGGTT |
| | | | CGATGATAGG AGAGCTAATG GTTTTCCAGT |
| | | | TCTTGTTGCT |
| | | | GATGTTGATG GTGTTGTTGC TGGATATGCT |
| | | | TCTTACGGAG |
| | | | TTTGGAGGGC TAAGTCTTCA TACAGACATA |
| | | | CTGTTGAAAA |
| | | | CTCAGTTTAC GTTCATGTTG ATCATCATAG |
| | | | GAGAGGTATT |
| | | | GCTACCGCTT TGATGACTGA ACTTATCGAG |
| | | | AGGGCTAGAG |
| | | | CTGGTGGTAT TCATGTTATC GTTGCTTCTG |
| | | | TTGAATCAAC |
| | | | TAATGCTACA TCAGTTGCTT TGCATGAGAG |
| | | | GTTCGGTTTC |
| | | | AGAATCGTTG CTCACATGCC TGAGGTTGGT |
| | | | AGGAAATTCG |
| | | | GAAGATGGCT TGATATGACA TATCTTCAAT |
| | | | TGACCCTTTG |
| | | | AGATCGTTCA AACATTTGGC AATAAAGTTT |
| | | | CTTAAGATTG |
| | | | AATCCTGTTG CCGGTCTTGC GATGATTATC |
| | | | ATATAATTTC |
| | | | TGTTGAATTA CGTTAAGCAT GTAATAATTA |
| | | | ACATGTAATG |
| | | | CATGACGTTA TTTATGAGAT GGGTTTTTAT |
| | | | GATTAGAGTC |
| | | | CCGCAATTAT ACATTTAATA CGCGATAGAA |
| | | | AACAAAATAT |
| | | | AGCGCGCAAA CTAGGATAAA TTATCGCGCG |
| | | | CGGTGTCATC |
| | | | TATGTTACTA GATCGGGCCC TCAGCGTGTC |
| | | | CTCTCCAAAT |
| | | | GAAATGAACT TCCTTATATA GAGGAAGGTC |
| | | | TTGCGAAGGA |
| | | | TAGTGGGATT GTGCGTCATC CCTTACGTCA |
| | | | GTGGAGATAT |
| | | | CACATCAATC CACTTGCTTT GAAGACGTGG |
| | | | TTGGAACGTC |
| | | | TTCTTTTTCC ACGATGCTCC TCGTGGGTGG |
| | | | GGGTCCATCT |
| | | | TTGGGACCAC TGTCGGCAGA GGCATCTTGA |
| | | | ACGATAGCCT |
| | | | TTCCTTTATC GCAATGATGG CATTTGTAGG |
| | | | TGCCACCTTC |
| | | | CTTTTCTACT GTCCTTTTGA TGAAGTGACA |
| | | | GATAGCTGGG |
| | | | CAATGGAATC CGAGGAGGTT TCCCGATATT |
| | | | ACCCTTTGTT |
| | | | GAAAAGTCTC AATAGCCCTT TGGTCTTCTG |
| | | | AGACTGTATC |
| | | | TTTGATATTC TTGGAGTAGA CGAGAGTGTC |
| | | | GTGCTCCACC |
| | | | ATGTTATCAC ATCAATCCAC TTGCTTTGAA |
| | | | GACGTGGTTG |
| | | | GAACGTCTTC TTTTTCCACG ATGCTCCTCG |
| | | | TGGGTGGGGG |
| | | | TCCATCTTTG GGACCACTGT CGGCAGAGGC |
| | | | ATCTTGAACG |
| | | | ATAGCCTTTC CTTTATCGCA ATGATGGCAT |
| | | | TTGTAGGTGC |
| | | | CACCTTCCTT TTCTACTGTC CTTTTGATGA |
| | | | AGTGACAGAT |
| | | | AGCTGGGCAA TGGAATCCGA GGAGGTTTCC |
| | | | CGATATTACC |
| | | | CTTTGTTGAA AAGTCTCAAC ACGCTGGAAT |
| | | | TCTAGTATAC |
| | | | TAAACCATGA AACTCAAAAA CCAAGACAAA |
| | | | CATCAGAGCT |
| | | | TCTCCTCAAA CGCTAAGGTG GATAAAATCA |
| | | | GCACAGACAG |
| | | | CCTCAAAAAC GAGACTGATA TTGAGCTTCA |
| | | | AAACATCAAT |
| | | | CATGAAGATT GTTTGAAGAT GTCAGAATAT |
| | | | GAGAACGTTG |
| | | | AGCCTTTCGT TTCAGCTTCT ACAATTCAAA |
| | | | CCGGTATTGG |
| | | | AATCGCTGGA AAGATTCTTG GTACACTTGG |
| | | | AGTTCCTTTC |
| | | | GCTGGACAAG TTGCTTCACT TTACTCTTTT |
| | | | ATTTTGGGTG |
| | | | AACTTTGGCC AAAGGGAAAA AATCAATGGG |
| | | | AAATTTTTAT |
| | | | GGAGCATGTT GAAGAGATTA TCAACCAAAA |
| | | | GATTTCTACT |
| | | | TATGCTAGAA ATAAGGCTTT GACTGATCTT |
| | | | AAAGGTTTGG |
| | | | GAGATGCTCT TGCTGTTTAC CATGATTCAT |
| | | | TGGAGTCTTG |
| | | | GGTTGGTAAC AGAAATAACA CAAGAGCTAG |
| | | | GTCTGTTGTT |
| | | | AGGTCACAAT ATATTGCTCT TGAATTGATG |
| | | | TTCGTTCAAA |
| | | | AATTGCCATC TTTTGCTGTT TCAGGAGAAG |
| | | | AGGTTCCTCT |
| | | | TTTGCCAATC TACGCTCAAG CTGCTAACCT |
| | | | TCATCTTTTG |
| | | | CTTTTGAGGG ATGCTTCAAT TTTCGGAAAG |
| | | | GAGTGGGGTC |
| | | | TTTCTTCTTC TGAAATCTCT ACTTTTTACA ATAGACAAGT |
| | | | TGAGAGGGCT GGAGATTATT CAGATCATTG |
| | | | CGTTAAATGG |
| | | | TACTCTACCG GTCTTAATAA CTTGAGAGGA |
| | | | ACTAACGCTG |
| | | | AATCATGGGT TAGGTACAAT CAATTCAGAA |
| | | | GGGATATGAC |
| | | | TCTTATGGTT CTTGATTTGG TTGCTTTGTT CCCTTCTTAT |
| | | | GATACACAAA TGTACCCAAT TAAGACTACA |
| | | | GCTCAACTTA |
| | | | CTAGAGAGGT TTATACAGAT GCTATCGGAA |
| | | | CCGTTCATCC |
| | | | TCATCCATCA TTCACTTCTA CCACTTGGTA |
| | | | CAATAACAAT |
| | | | TTTCTGCTAT TGAGGCTGCT GTTGTTAGGA |
| | | | ACCCACATCT |
| | | | TTTGGATTTC TTGGAACAAG TTACAATCTA TTCTCTTCTT |
| | | | TCTAGGTGGT CAAACACCCA ATACATGAAT |
| | | | ATGTGGGGTG |
| | | | GACATAAGTT GGAGTTCAGA ACCATTGGTG |
| | | | GAACTTTGAA |
| | | | TATCTCTACC CAAGGTTCAA CCAACACTTC |
| | | | TATTAATCCT |
| | | | GTTACACTTC CATTCACCTC AAGAGATGTT |
| | | | TATAGGACTG |
| | | | AATCTTTGGC TGGTCTTAAC TTGTTTCTTA |
| | | | CACAACCTGT |
| | | | TAATGGTGTT CCTAGAGTTG ATTTCCATTG |
| | | | GAAATTTGTT |
| | | | ACTCATCCTA TCGCTTCAGA TAACTTCTAC |
| | | | TACCCAGGAT |
| | | | ACGCTGGTAT CGGAACACAA CTTCAAGATT |
| | | | CTGAAAACGA |
| | | | GTTGCCTCCA GAGGCTACCG GTCAACCTAA |
| | | | TTATGAATCA |
| | | | TACTCTCATA GGCTTTCACA TATTGGATTG |
| | | | ATCTCAGCTT |
| | | | CTCATGTTAA GGCTCTTGTT TATTCATGGA |
| | | | CACATAGGTC |
| | | | TGCTGATAGG ACAAACACCA TTGAACCTAA |
| | | | TTCTATTACC |
| | | | CAAATCCCTC TTGTTAAGGC TTTCAATCTT TCTTCTGGTG |
| | | | CTGCTGTTGT TAGAGGTCCT GGTTTCACTG |
| | | | GTGGAGATAT |
| | | | TCTTAGAAGG ACTAATACAG GTACTTTCGG |
| | | | AGATATTAGG |
| | | | GTTAACATCA ATCCTCCATT TGCTCAAAGA |
| | | | TATAGGGTTA |
| | | | GAATCAGGTA CGCTTCAACA ACCGATTTGC |
| | | | AATTCCATAC |
| | | | TTCTATTAAC GGAAAGGCTA TCAACCAAGG |
| | | | AAATTTTTCT |
| | | | GCTACTATGA ATAGAGGTGA GGATCTTGAT |
| | | | TATAAGACTT |
| | | | TCAGGACAGT TGGTTTCACT ACACCTTTCT |
| | | | CATTTTTGGA |
| | | | TGTTCAATCT ACTTTCACAA TTGGTGCTTG |
| | | | GAACTTTTCA |
| | | | TCTGGAAACG AAGTTTACAT CGATAGAATC |
| | | | GAATTTGTTC |
| | | | CTGTGGAGGT TACTTATGAA GCTGAGTACG |
| | | | ATTTCGAAAA |
| | | | GGCTCAAGAG AAAGTTACCG CTCTTTTTAC |
| | | | CTCAACTAAC |
| | | | CCTAGAGGTC TTAAGACTGA TGTTAAAGAT |
| | | | TACCATATCG |
| | | | ATCAAGTTTC TAATCTTGTT GAATCATTGT |
| | | | CTGATGAGTT |
| | | | TTACCTTGAT GAAAAGAGGG AATTGTTTGA |
| | | | AATCGTGAAG |
| | | | TATGCCAAGC AACTCCACAT TGAAAGAAAT |
| | | | ATGTAAGATC |
| | | | GTTCAAACAT TTGGCAATAA AGTTTCTTAA |
| | | | GATTGAATCC |
| | | | TGTTGCCGGT CTTGCGATGA TTATCATATA |
| | | | ATTTCTGTTG |
| | | | AATTACGTTA AGCATGTAAT AATTAACATG |
| | | | TAATGCATGA |
| | | | CGTTATTTAT GAGATGGGTT TTTATGATTA |
| | | | GAGTCCCGCA |
| | | | ATTATACATT TAATACGCGA TAGAAAACAA |
| | | | AATATAGCGC |
| | | | GCAAACTAGG ATAAATTATC GCGCGCGGTG |
| | | | TCATCTATGT |
| | | | TACTAGATCT CGCGATCAGC GTGTCCTCTC |
| | | | CAAATGAAAT |
| | | | GAACTTCCTT ATATAGAGGA AGGTCTTGCG |
| | | | AAGGATAGTG |
| | | | GGATTGTGCG TCATCCCTTA CGTCAGTGGA |
| | | | GATATCACAT |
| | | | CAATCCACTT GCTTTGAAGA CGTGGTTGGA |
| | | | ACGTCTTCTT |
| | | | TTTCCACGAT GCTCCTCGTG GGTGGGGGTC |
| | | | CATCTTTGGG |
| | | | ACCACTGTCG GCAGAGGCAT CTTGAACGAT |
| | | | AGCCTTTCCT |
| | | | TTATCGCAAT GATGGCATTT GTAGGTGCCA |
| | | | CCTTCCTTTT |
| | | | CTACTGTCCT TTTGATGAAG TGACAGATAG |
| | | | CTGGGCAATG |
| | | | GAATCCGAGG AGGTTTCCCG ATATTACCCT |
| | | | TTGTTGAAAA |
| | | | GTCTCAATAG CCCTTTGGTC TTCTGAGACT |
| | | | GTATCTTTGA |
| | | | TATTCTTGGA GTAGACGAGA GTGTCGTGCT |
| | | | CCACCATGTT |
| | | | ATCACATCAA TCCACTTGCT TTGAAGACGT |
| | | | GGTTGGAACG |
| | | | TCTTCTTTTT CCACGATGCT CCTCGTGGGT |
| | | | GGGGGTCCAT |
| | | | CTTTGGGACC ACTGTCGGCA GAGGCATCTT |
| | | | GAACGATAGC |
| | | | CTTTCCTTTA TCGCAATGAT GGCATTTGTA |
| | | | GGTGCCACCT |
| | | | TCCTTTTCTA CTGTCCTTTT GATGAAGTGA |
| | | | CAGATAGCTG |
| | | | GGCAATGGAA TCCGAGGAGG TTTCCCGATA |
| | | | TTACCCTTTG |
| | | | TTGAAAAGTC TCAACACGCT GGAATTCTAG |
| | | | TATACTAAAC |
| | | | CATGAACCCT TATCAGAACA AGAACGAGTA |
| | | | TGAGATTTTT |
| | | | AACGCTCCAT CCAACGGCTT TAGCAAGTCA |
| | | | AACAACTACA |
| | | | GCAGGTATCC ACTTGCTAAC AAGCCTAATC |
| | | | AACCATTGAA |
| | | | GAATACCAAC TACAAGGATT GGCTTAATGT |
| | | | TTGCCAAGAT |
| | | | AATCAACAAT ATGGTAACAA TGCTGGAAAC |
| | | | TTCGCTTCTT |
| | | | CAGAGACAAT TGTTGGTGTT TCTGCTGGTA |
| | | | TCATCGTTGT |
| | | | TGGTACAATG CTTGGTGCTT TTGCTGCTCC TGTTTTGGCT |
| | | | GCTGGTATTA TTTCTTTTGG AACATTGCTT CCTATTTTCT |
| | | | GGCAAGGTTC TGATCCTGCT AACGTTTGGC |
| | | | AAGATTTGTT |
| | | | GAATATCGGT GGTAGGCCAA TCCAAGAGAT |
| | | | TGATAAGAAT |
| | | | ATCATTAACG TTTTGACCTC TATCGTTACA |
| | | | CCTATTAAGA |
| | | | ACCAACTTGA TAAGTACCAA GAATTTTTCG |
| | | | ATAAATGGGA |
| | | | ACCTGCTAGG ACACATGCTA ACGCTAAGGC |
| | | | TGTTCATGAT |
| | | | CTTTTCACCA CCTTGGAGCC AATTATCGAT |
| | | | AAGGATTTGG |
| | | | ATATGCTTAA GAACAACGCT TCATACAGAA |
| | | | TCCCTACTCT |
| | | | TCCAGCTTAT GCTCAAATTG CTACATGGCA |
| | | | TCTTAATTTG |
| | | | CTTAAGCATG CTGCTACTTA TTACAATATC |
| | | | TGGCTTCAAA |
| | | | ACCAAGGAAT CAATCCATCA ACTTTTAATT |
| | | | CATCTAACTA |
| | | | CTATCAAGGT TATCTTAAGA GGAAAATTCA |
| | | | AGAGTATACT |
| | | | GATTACTGCA TTCAAACTTA TAACGCTGGA |
| | | | CTTACCATGA |
| | | | TTAGGACAAA TACCAACGCT ACTTGGAATA |
| | | | TGTATAATAC |
| | | | CTACAGGCTT GAGATGACTT TGACCGTTTT |
| | | | GGATCTTATC |
| | | | GCTATCTTCC CAAATTATGA TCCAGAGAAG |
| | | | TACCCTATCG |
| | | | GTGTTAAGTC AGAACTTATT AGAGAAGTTT |
| | | | ACACTAATGT |
| | | | TAACTCTGAT ACTTTTAGAA CAATCACAGA |
| | | | GTTGGAAAAC |
| | | | GGTCTTACAA GAAATCCAAC ACTTTTTACC |
| | | | TGGATCAACC |
| | | | AAGGAAGATT CTACACTAGG AACTCTAGGG |
| | | | ATATTCTTGA |
| | | | TCCTTACGAT ATTTTCTCAT TCACAGGAAA |
| | | | TCAAATGGCT |
| | | | TTTACTCATA CAAACGATGA TAGGAATATC |
| | | | ATCTGGGGTG |
| | | | CTGTTCATGG AAACATCATC TCACAAGATA |
| | | | CATCAAAGGT |
| | | | TTTCCCATTT TACAGGAACA AGCCTATCGA |
| | | | TAAGGTTGAG |
| | | | ATCGTTAGGC ATAGGGAGTA TTCTGATATT |
| | | | ATCTATGAGA |
| | | | TGATCTTCTT TTCAAACTCA TCTGAAGTTT TCAGATATTC |
| | | | ATCTAACTCT ACTATCGAAA ACAATTACAA |
| | | | AAGAACAGAT |
| | | | TCTTATATGA TTCCTAAGCA AACTTGGAAG |
| | | | AACAAGGAAT |
| | | | ACGGACATAC TCTTTCTTAT ATCAAGACAG |
| | | | ATAACTACAT |
| | | | CTTCTCAGTT GTTAGAGAGA GGAGAAGGGT |
| | | | TGCTTTCTCA |
| | | | TGGACTCATA CTTCTGTTGA TTTTCAAAAC |
| | | | ACTATTGATT |
| | | | TGGATAACAT TACCCAAATT CATGCTCTTA |
| | | | AGGCTTTGAA |
| | | | GGTTTCTTCA GATTCTAAAA TCGTTAAGGG |
| | | | TCCTGGACAT |
| | | | ACTGGAGGAG ATTTGGTTAT TTTGAAGGAT |
| | | | TCTATGGATT |
| | | | TTAGAGTTAG ATTTCTTAAG AACGTTTCAA |
| | | | GGCAATACCA |
| | | | AGTTAGGATT AGATATGCTA CCAACGCTCC |
| | | | TAAGACAACT |
| | | | GTTTTCTTGA CTGGTATTGA TACAATCTCA |
| | | | GTTGAACTTC |
| | | | CATCTACAAC CTCTAGGCAA AATCCAAATG |
| | | | CTACTGATCT |
| | | | TACTTACGCT GATTTCGGAT ACGTTACCTT |
| | | | TCCAAGAACC |
| | | | GTTCCTAACA AAACTTTCGA GGGTGAAGAT |
| | | | ACACTTTTGA |
| | | | TGACATTGTA TGGTACACCT AACCATTCTT |
| | | | ATAACATCTA |
| | | | CATTGATAAG ATCGAGTTTA TCCCAATTAC |
| | | | TCAATCTGTT |
| | | | TTGGATTATA CAGAAAAGCA GAATATCGAG |
| | | | AAGACCCAGA |
| | | | AGATTGTGAA TGATTTGTTT GTGAACTGAG |
| | | | ATCGTTCAAA |
| | | | CATTTGGCAA TAAAGTTTCT TAAGATTGAA |
| | | | TCCTGTTGCC |
| | | | GGTCTTGCGA TGATTATCAT ATAATTTCTG |
| | | | TTGAATTACG |
| | | | TTAAGCATGT AATAATTAAC ATGTAATGCA |
| | | | TGACGTTATT |
| | | | TATGAGATGG GTTTTTATGA TTAGAGTCCC |
| | | | GCAATTATAC |
| | | | ATTTAATACG CGATAGAAAA CAAAATATAG |
| | | | CGCGCAAACT |
| | | | AGGATAAATT ATCGCGCGCG GTGTCATCTA |
| | | | TGTTACTAGA |
| | | | TCGGTACC |
| 15 | DNA | Artificial | ATGTTGATTAGGGAT GCTGTTCCTGGAGAT |
| | | Sequence | TTGCCTGGTATTCTT GAAATCCATAATGAG |
| | | | GCTATTGCTAACTCT ACTGCTATCTGGGAT |
| | | | GAAACACCTGCTGAT TTGGATGAGAGAAGG |
| | | | AGATGGTTCGATGAT AGGAGAGCTAATGGT |
| | | | TTTCCAGTTCTTGTT GCTGATGTTGATGGT |
| | | | GTTGTTGCTGGATAT GCTTCTTACGGAGTT |
| | | | TGGAGGGCTAAGTCTTCATACAGACATACT |
| | | | GTTGAAAACTCAGTT TACGTTCATGTTGAT |
| | | | CATCATAGGAGAGGT ATTGCTACCGCTTTG |
| | | | ATGACTGAACTTATC GAGAGGGCTAGAGCT |
| | | | GGTGGTATTCATGTT ATCGTTGCTTCTGTT |
| | | | GAATCAACTAATGCTACATCAGTTGCTTTG |
| | | | CATGAGAGGTTCGGT TTCAGAATCGTTGCT |
| | | | CACATGCCTGAGGTT GGTAGGAAATTCGGA |
| | | | AGATGGCTTGATATG ACATATCTTCAATTG |
| | | | ACCCTTTGA |
| 16 | DNA | Artificial | ATGAAACTCAAAAAC CAAGACAAACATCAG |
| | | Sequence | AGCTTCTCCTCAAAC GCTAAGGTGGATAAA |
| | | | ATCAGCACAGACAGCCTCAAAAACGAGACT |
| | | | GATATTGAGCTTCAA AACATCAATCATGAA |
| | | | GATTGTTTGAAGATG TCAGAATATGAGAAC |
| | | | GTTGAGCCTTTCGTT TCAGCTTCTACAATT |
| | | | CAAACCGGTATTGGA ATCGCTGGAAAGATT |
| | | | CTTGGTACACTTGGAGTTCCTTTCGCTGGA |
| | | | CAAGTTGCTTCACTT TACTCTTTTATTTTG |
| | | | GGTGAACTTTGGCCA AAGGGAAAAAATCAA |
| | | | TGGGAAATTTTTATG GAGCATGTTGAAGAG |
| | | | ATTATCAACCAAAAG ATTTCTACTTATGCT |
| | | | AGAAATAAGGCTTTGACTGATCTTAAAGGT |
| | | | TTGGGAGATGCTCTT GCTGTTTACCATGAT |
| | | | TCATTGGAGTCTTGG GTTGGTAACAGAAAT |
| | | | AACACAAGAGCTAGG TCTGTTGTTAGGTCA |
| | | | CAATATATTGCTCTT GAATTGATGTTCGTT |
| | | | CAAAAATTGCCATCTTTTGCTGTTTCAGGA |
| | | | GAAGAGGTTCCTCTT TTGCCAATCTACGCT |
| | | | CAAGCTGCTAACCTT CATCTTTTGCTTTTG |
| | | | AGGGATGCTTCAATT TTCGGAAAGGAGTGG |
| | | | GGTCTTTCTTCTTCT GAAATCTCTACTTTT |
| | | | TACAATAGACAAGTTGAGAGGGCTGGAGAT |
| | | | TATTCAGATCATTGC GTTAAATGGTACTCT |
| | | | ACCGGTCTTAATAAC TTGAGAGGAACTAAC |
| | | | GCTGAATCATGGGTT AGGTACAATCAATTC |
| | | | AGAAGGGATATGACT CTTATGGTTCTTGAT |
| | | | TTGGTTGCTTTGTTCCCTTCTTATGATACA |
| | | | CAAATGTACCCAATT AAGACTACAGCTCAA |
| | | | CTTACTAGAGAGGTT TATACAGATGCTATC |
| | | | GGAACCGTTCATCCT CATCCATCATTCACT |
| | | | TCTACCACTTGGTAC AATAACAATGCTCCT |
| | | | TCATTTTCTGCTATTGAGGCTGCTGTTGTT |
| | | | AGGAACCCACATCTT TTGGATTTCTTGGAA |
| | | | CAAGTTACAATCTAT TCTCTTCTTTCTAGG |
| | | | TGGTCAAACACCCAA TACATGAATATGTGG |
| | | | GGTGGACATAAGTTG GAGTTCAGAACCATT |
| | | | GGTGGAACTTTGAATATCTCTACCCAAGGT |
| | | | TCAACCAACACTTCT ATTAATCCTGTTACA |
| | | | CTTCCATTCACCTCA AGAGATGTTTATAGG |
| | | | ACTGAATCTTTGGCT GGTCTTAACTTGTTT |
| | | | CTTACACAACCTGTT AATGGTGTTCCTAGA |
| | | | GTTGATTTCCATTGGAAATTTGTTACTCAT |
| | | | CCTATCGCTTCAGAT AACTTCTACTACCCA |
| | | | GGATACGCTGGTATC GGAACACAACTTCAA |
| | | | GATTCTGAAAACGAG TTGCCTCCAGAGGCT |
| | | | ACCGGTCAACCTAAT TATGAATCATACTCT |
| | | | CATAGGCTTTCACATATTGGATTGATCTCA |
| | | | GCTTCTCATGTTAAG GCTCTTGTTTATTCA |
| | | | TGGACACATAGGTCT GCTGATAGGACAAAC |
| | | | ACCATTGAACCTAAT TCTATTACCCAAATC |
| | | | CCTCTTGTTAAGGCT TTCAATCTTTCTTCT |
| | | | GGTGCTGCTGTTGTTAGAGGTCCTGGTTTC |
| | | | ACTGGTGGAGATATT CTTAGAAGGACTAAT |
| | | | ACAGGTACTTTCGGA GATATTAGGGTTAAC |
| | | | ATCAATCCTCCATTT GCTCAAAGATATAGG |
| | | | GTTAGAATCAGGTAC GCTTCAACAACCGAT |
| | | | TTGCAATTCCATACTTCTATTAACGGAAAG |
| | | | GCTATCAACCAAGGA AATTTTTCTGCTACT |
| | | | ATGAATAGAGGTGAG GATCTTGATTATAAG |
| | | | ACTTTCAGGACAGTT GGTTTCACTACACCT |
| | | | TTCTCATTTTTGGAT GTTCAATCTACTTTC |
| | | | ACAATTGGTGCTTGGAACTTTTCATCTGGA |
| | | | AACGAAGTTTACATC GATAGAATCGAATTT |
| | | | GTTCCTGTGGAGGTT ACTTATGAAGCTGAG |
| | | | TACGATTTCGAAAAG GCTCAAGAGAAAGTT |
| | | | ACCGCTCTTTTTACC TCAACTAACCCTAGA |
| | | | GGTCTTAAGACTGATGTTAAAGATTACCAT |
| | | | ATCGATCAAGTTTCT AATCTTGTTGAATCA |
| | | | TTGTCTGATGAGTTT TACCTTGATGAAAAG |
| | | | AGGGAATTGTTTGAA ATCGTGAAGTATGCC |
| | | | AAGCAACTCCACATT GAAAGAAATATGTAA |
| 17 | DNA | Artificial | ATGAACCCTTATCAG AACAAGAACGAGTAT |
| | | Sequence | GAGATTTTTAACGCT CCATCCAACGGCTTT |
| | | | AGCAAGTCAAACAACTACAGCAGGTATCCA |
| | | | CTTGCTAACAAGCCT AATCAACCATTGAAG |
| | | | AATACCAACTACAAG GATTGGCTTAATGTT |
| | | | TGCCAAGATAATCAA CAATATGGTAACAAT |
| | | | GCTGGAAACTTCGCT TCTTCAGAGACAATT |
| | | | GTTGGTGTTTCTGCTGGTATCATCGTTGTT |
| | | | GGTACAATGCTTGGT GCTTTTGCTGCTCCT |
| | | | GTTTTGGCTGCTGGT ATTATTTCTTTTGGA |
| | | | ACATTGCTTCCTATT TTCTGGCAAGGTTCT |
| | | | GATCCTGCTAACGTT TGGCAAGATTTGTTG |
| | | | AATATCGGTGGTAGGCCAATCCAAGAGATT |
| | | | GATAAGAATATCATT AACGTTTTGACCTCT |
| | | | ATCGTTACACCTATT AAGAACCAACTTGAT |
| | | | AAGTACCAAGAATTTTTCGATAAATGGGAA |
| | | | CCTGCTAGGACACAT GCTAACGCTAAGGCT |
| | | | GTTCATGATCTTTTCACCACCTTGGAGCCA |
| | | | ATTATCGATAAGGAT TTGGATATGCTTAAG |
| | | | AACAACGCTTCATAC AGAATCCCTACTCTT |
| | | | CCAGCTTATGCTCAA ATTGCTACATGGCAT |
| | | | CTTAATTTGCTTAAG CATGCTGCTACTTAT |
| | | | TACAATATCTGGCTTCAAAACCAAGGAATC |
| | | | AATCCATCAACTTTT AATTCATCTAACTAC |
| | | | TATCAAGGTTATCTT AAGAGGAAAATTCAA |
| | | | GAGTATACTGATTAC TGCATTCAAACTTAT |
| | | | AACGCTGGACTTACC ATGATTAGGACAAAT |
| | | | ACCAACGCTACTTGGAATATGTATAATACC |
| | | | TACAGGCTTGAGATG ACTTTGACCGTTTTG |
| | | | GATCTTATCGCTATC TTCCCAAATTATGAT |
| | | | CCAGAGAAGTACCCT ATCGGTGTTAAGTCA |
| | | | GAACTTATTAGAGAA GTTTACACTAATGTT |
| | | | AACTCTGATACTTTTAGAACAATCACAGAG |
| | | | TTGGAAAACGGTCTT ACAAGAAATCCAACA |
| | | | CTTTTTACCTGGATC AACCAAGGAAGATTC |
| | | | TACACTAGGAACTCT AGGGATATTCTTGAT |
| | | | CCTTACGATATTTTC TCATTCACAGGAAAT |
| | | | CAAATGGCTTTTACTCATACAAACGATGAT |
| | | | AGGAATATCATCTGG GGTGCTGTTCATGGA |
| | | | AACATCATCTCACAA GATACATCAAAGGTT |
| | | | TTCCCATTTTACAGG AACAAGCCTATCGAT |
| | | | AAGGTTGAGATCGTT AGGCATAGGGAGTAT |
| | | | TCTGATATTATCTATGAGATGATCTTCTTT |
| | | | TCAAACTCATCTGAA GTTTTCAGATATTCA |
| | | | TCTAACTCTACTATC GAAAACAATTACAAA |
| | | | AGAACAGATTCTTATATGATTCCTAAGCAA |
| | | | ACTTGGAAGAACAAG GAATACGGACATACT |
| | | | CTTTCTTATATCAAGACAGATAACTACATC |
| | | | TTCTCAGTTGTTAGA GAGAGGAGAAGGGTT |
| | | | GCTTTCTCATGGACT CATACTTCTGTTGAT |
| | | | TTTCAAAACACTATT GATTTGGATAACATT |
| | | | ACCCAAATTCATGCT CTTAAGGCTTTGAAG |
| | | | GTTTCTTCAGATTCTAAAATCGTTAAGGGT |
| | | | CCTGGACATACTGGA GGAGATTTGGTTATT |
| | | | TTGAAGGATTCTATG GATTTTAGAGTTAGA |
| | | | TTTCTTAAGAACGTT TCAAGGCAATACCAA |
| | | | GTTAGGATTAGATAT GCTACCAACGCTCCT |
| | | | AAGACAACTGTTTTCTTGACTGGTATTGAT |
| | | | ACAATCTCAGTTGAA CTTCCATCTACAACC |
| | | | TCTAGGCAAAATCCA AATGCTACTGATCTT |
| | | | ACTTACGCTGATTTC GGATACGTTACCTTT |
| | | | CCAAGAACCGTTCCT AACAAAACTTTCGAG |
| | | | GGTGAAGATACACTTTTGATGACATTGTAT |
| | | | GGTACACCTAACCAT TCTTATAACATCTAC |
| | | | ATTGATAAGATCGAG TTTATCCCAATTACT |
| | | | CAATCTGTTTTGGATTATACAGAAAAGCAG |
| | | | AATATCGAGAAGACC CAGAAGATTGTGAAT |
| | | | GATTTGTTTGTGAACTGA |
| 18 | DNA | Artificial | CCCGGGTCAG CGTGTCCTCT CCAAATGAAA |
| | | Sequence | TGAACTTCCT TATATAGAGG AAGGTCTTGC |
| | | | GAAGGATAGT GGGATTGTGC GTCATCCCTT |
| | | | ACGTCAGTGG AGATATCACA TCAATCCACT |
| | | | TGCTTTGAAG ACGTGGTTGG AACGTCTTCT |
| | | | TTTTCCACGA |
| | | | TGCTCCTCGT GGGTGGGGGT CCATCTTTGG |
| | | | GACCACTGTC GGCAGAGGCA TCTTGAACGA |
| | | | TAGCCTTTCC TTTATCGCAA TGATGGCATT |
| | | | TGTAGGTGCC ACCTTCCTTT TCTACTGTCC |
| | | | TTTTGATGAA GTGACAGATA GCTGGGCAAT |
| | | | GGAATCCGAG GAGGTTTCCC GATATTACCC |
| | | | TTTGTTGAAA AGTCTCAATA GCCCTTTGGT |
| | | | CTTCTGAGAC TGTATCTTTG ATATTCTTGG |
| | | | AGTAGACGAG AGTGTCGTGC TCCACCATGT |
| | | | TATCACATCA ATCCACTTGC TTTGAAGACG |
| | | | TGGTTGGAAC GTCTTCTTTT TCCACGATGC |
| | | | TCCTCGTGGG TGGGGGTCCA TCTTTGGGAC |
| | | | CACTGTCGGC AGAGGCATCT TGAACGATAG |
| | | | CCTTTCCTTT ATCGCAATGA TGGCATTTGT |
| | | | AGGTGCCACC TTCCTTTTCT ACTGTCCTTT |
| | | | TGATGAAGTG ACAGATAGCT GGGCAATGGA |
| | | | ATCCGAGGAG GTTTCCCGAT ATTACCCTTT |
| | | | GTTGAAAAGT CTCAACACGC TGGAATTCTA |
| | | | GTATACTAAA CC |
| 19 | DNA | Synthetic | CCCAGAAGAT TGTGAATGAT TTGTTTGTGA |
| | | Sequence | ACTGAGATCG TTCAAACATT TGGCAATAAA |
| | | (1-294) + | GTTTCTTAAG ATTGAATCCT GTTGCCGGTC |
| | | Gossypium | TTGCGATGAT TATCATATAA TTTCTGTTGA |
| | | hirsutum | ATTACGTTAA GCATGTAATA ATTAACATGT |
| | | genome | AATGCATGAC GTTATTTATG AGATGGGTTT |
| | | (294-537) | TTATGATTAG AGTCCCGCAA TTATACATTT |
| | | | AATACGCGAT AGAAAACAAA ATATAGCGCG |
| | | | CAAACTAGGA TAAATTATCG CGCGCGGTGT |
| | | | CATCTATGTT ACTAGATCGG TACCGTAAAC |
| | | | CTAAGAGAAA AGAGCGTTTA CAGCTGTGAC |
| | | | AGGAATCTTG GCTGGTTCAA ATCGATCCGC |
| | | | CTCACTTGAA GACACCCAGC GTTCTATCCC |
| | | | CATCGGGACG TTGGCCGCCA CACTTACTAC |
| | | | TACTGGACTC TATTTAGTCT CCGTCTTACT |
| | | | TTTTGGAGCT GTTGCCACCA GAGACAAGCT |
| | | | TGACTGACAG GCTACTTACG GCTACAATTG |
| | | | CTTGGCCTTT TCCAGCTATC ATTCATA |
| 20 | DNA | Artificial | CCCAGAAGATTGTGAATGATTTGT |
| | | Sequence | |
| 21 | DNA | Gossypium | TATGAATGATAGCTGGAAAAGGCC |
| | | hirsutum | |

What is claimed is:

1. A profusion of cassettes having recombinant polynucleotide sequences comprising:
a triple gene (SEQ ID NO: 14) with a 5′ end attached through a promoter joined to an un-translated enhancer (intron) sequence and a 3′ end attached to a NOS terminator sequence, for encoding the polynucleotide sequences;
wherein the triple gene comprises one Re-PAT herbicidal gene comprising the nucleotide sequence of SEQ ID NO: 11 and two B. thuringiensis δ-endotoxin Cry10A and Cry11a12 genes comprising the nucleotide sequence of SEQ ID NO: 12 SEQ ID NO: 13 respectively; and
wherein the genes encode herbicidal and insecticidal toxin proteins in a transgenic plant, resulting in decreased resistance development against insecticidal toxin proteins and increased efficacy against the insect mortality.

2. The profusion of cassettes according to claim 1, wherein a first cassette, a second cassette, and a third cassette are located within a T-DNA region of a vector flanked by a left and a right border sequence.

3. The profusion of cassettes according to claim 2, wherein the first cassette coding the Re-PAT gene comprising the nucleotide sequence of SEQ ID NO: 11 operably linked to the promoter, the un-translated enhancer sequence at a 5′ end and the NOS terminator sequence tagged to a 3′ end of the Re-PAT gene.

4. The profusion of cassettes according to claim 2, wherein the second cassette coding insecticidal Cry10A gene comprising the nucleotide sequence of SEQ ID NO: 12 operably linked to the promoter, the un-translated enhancer sequence at a 5′ end and the NOS terminator sequence tagged to a 3′ end of the gene Cry10A.

5. The profusion of cassettes according to claim 2, wherein the third cassette coding the insecticidal Cry11a12 gene comprising the nucleotide sequence of SEQ ID NO: 13 operably linked to the promoter, the un-translated enhancer sequence at a 5′ end and the NOS terminator sequence tagged to a 3′ end of the Cry11a12 gene.

6. The profusion of cassettes according to claim 1, wherein the 5′ end of each gene, the Re-PAT, the Cry10A and the Cry11a12 gene in the transgenic plants is attached with the un-translated enhancer sequence (intron) comprising 28 nucleotides of SEQ ID NO: 14 starting from the 685^{th} nucleotide to the 712^{th} nucleotide.

7. The profusion of cassettes according to claim 1, wherein the promoter is Cauliflower mosaic virus (CaMV35S).

8. The profusion of cassettes according to claim 1, wherein the profusion of cassettes comprising the nucleotide sequence of SEQ ID NO: 14 is present in the transgenic plant or the part of the transgenic plant.

9. The profusion of cassettes according to claim 1, wherein the transgenic plant is a monocot plant selected from the group consisting of maize, sugarcane, and wheat.

10. The profusion of cassettes according to claim 1, wherein the transgenic plant is a dicot plant selected from the group consisting of cotton, potato and tomato.

11. The profusion of cassettes according to claim 10, wherein the dicot plant is the cotton plant.

12. The profusion of cassettes according to claim 1, wherein the profusion of cassettes are located at SEQ ID NO: 19 having a forward primer of SEQ ID NO: 20 and a reverse primer of SEQ ID NO: 21 for identification.

13. A recombinant DNA molecule comprising a polynucleotide sequence selected from the group consisting of SEQ ID NOS: 1-14, SEQ ID NO: 19, SEQ ID NO: 20, and SEQ ID NO: 21 and complements thereof.

14. The recombinant DNA molecule of claim 13, wherein the DNA molecule comprises at least one of SEQ ID NOS: 1-14, SEQ ID NO: 19, SEQ ID NO: 20, and SEQ ID NO: 21, and complements thereof in at least a portion of a transgenic cotton plant selected from the group consisting of a cell, seed and part.

15. The recombinant DNA molecule of claim 13, wherein the DNA molecule comprises nucleotide SEQ ID NO: 19 in at least a portion of a transgenic cotton plant selected from the group consisting of a cell, seed and part.

16. The recombinant DNA molecule of claim 13, wherein the DNA molecule comprises at least one of SEQ ID NOS: 1-13 in at least a portion of a transgenic cotton plant selected from the group consisting of a cell, seed and part.

17. The recombinant DNA molecule of claim 13, wherein the DNA molecule comprises at least one of SEQ ID NOS: 11-13 in at least a portion of a transgenic cotton plant selected from the group consisting of a cell, seed and part.

18. The transgenic cotton plant, seed, cells or plant part thereof of claim 14, wherein an amplicon comprises the DNA molecule having the sequence of at least one of SEQ ID NOS: 1-10.

19. The transgenic cotton plant, seed, cells or plant part thereof of claim 14, wherein an amplicon comprises the DNA molecule having the sequence of SEQ ID NO: 14.


SEQUENCE LISTING (24 sequences)

SEQ ID NO: 1 - DNA, 23 bp, Artificial Sequence
  Description of Artificial Sequence Synthetic FBG cotton nucleotide motif
atgttgattagggatgctgttcc

SEQ ID NO: 2 - DNA, 23 bp, Artificial Sequence
  Description of Artificial Sequence Synthetic FBG cotton nucleotide motif
atatcaagccatcttccgaattt

SEQ ID NO: 3 - DNA, 26 bp, Artificial Sequence
  Description of Artificial Sequence Synthetic FBG cotton nucleotide motif
atgctagaaataaggctttgactgat

SEQ ID NO: 4 - DNA, 26 bp, Artificial Sequence
  Description of Artificial Sequence Synthetic FBG cotton nucleotide motif
aatcgtactcagcttcataagtaacc

SEQ ID NO: 5 - DNA, 27 bp, Artificial Sequence
  Description of Artificial Sequence Synthetic FBG cotton nucleotide motif
atcagaacaagaacgagtatgagattt

SEQ ID NO: 6 - DNA, 27 bp, Artificial Sequence
  Description of Artificial Sequence Synthetic FBG cotton nucleotide motif
cgttggtagcatatctaatcctaactt

SEQ ID NO: 7 - DNA, 23 bp, Artificial Sequence
  Description of Artificial Sequence Synthetic FBG cotton nucleotide motif
cgctggaattctagtagatgctg

SEQ ID NO: 8 - DNA, 23 bp, Artificial Sequence
  Description of Artificial Sequence Synthetic FBG cotton nucleotide motif
gctctagccctctcgataagttc

SEQ ID NO: 9 - DNA, 26 bp, Artificial Sequence
  Description of Artificial Sequence Synthetic FBG cotton nucleotide motif
ttaaggctcttgtttattcatggaca

SEQ ID NO: 10 - DNA, 26 bp, Artificial Sequence
  Description of Artificial Sequence Synthetic FBG cotton nucleotide motif
gttctgataagggttcatggtttagt

SEQ ID NO: 11 - DNA, 489 bp, Artificial Sequence
  Description of Artificial Sequence Synthetic FBG cotton nucleotide motif
atgttgattagggatgctgttcctggagatttgcctggtattcttgaaatccataatgaggctattgctaactctactgctatctgggatgaaacacctgctgatttggatgagagaaggagatggttcgatgataggagagctaatggttttccagttcttgttgctgatgttgatggtgttgttgctggatatgcttcttacggagtttggagggctaagtcttcatacagacatactgttgaaaactcagtttacgttcatgttgatcatcataggagaggtattgctaccgctttgatgactgaacttatcgagagggctagagctggtggtattcatgttatcgttgcttctgttgaatcaactaatgctacatcagttgctttgcatgagaggttcggtttcagaatcgttgctcacatgcctgaggttggtaggaaattcggaagatggcttgatatgacatatcttcaattgaccctttga

SEQ ID NO: 12 - DNA, 2160 bp, Artificial Sequence
  Description of Artificial Sequence Synthetic FBG cotton nucleotide motif
atgaaactcaaaaaccaagacaaacatcagagcttctcctcaaacgctaaggtggataaaatcagcacagacagcctcaaaaacgagactgatattgagcttcaaaacatcaatcatgaagattgtttgaagatgtcagaatatgagaacgttgagcctttcgtttcagcttctacaattcaaaccggtattggaatcgctggaaagattcttggtacacttggagttcctttcgctggacaagttgcttcactttactcttttattttgggtgaactttggccaaagggaaaaaatcaatgggaaatttttatggagcatgttgaagagattatcaaccaaaagatttctacttatgctagaaataaggctttgactgatcttaaaggtttgggagatgctcttgctgtttaccatgattcattggagtcttgggttggtaacagaaataacacaagagctaggtctgttgttaggtcacaatatattgctcttgaattgatgttcgttcaaaaattgccatcttttgctgtttcaggagaagaggttcctcttttgccaatctacgctcaagctgctaaccttcatcttttgcttttgagggatgcttcaattttcggaaaggagtggggtctttcttcttctgaaatctctactttttacaatagacaagttgagagggctggagattattcagatcattgcgttaaatggtactctaccggtcttaataacttgagaggaactaacgctgaatcatgggttaggtacaatcaattcagaagggatatgactcttatggttcttgatttggttgctttgttcccttcttatgatacacaaatgtacccaattaagactacagctcaacttactagagaggtttatacagatgctatcggaaccgttcatcctcatccatcattcacttctaccacttggtacaataacaatgctccttcattttctgctattgaggctgctgttgttaggaacccacatcttttggatttcttggaacaagttacaatctattctcttctttctaggtggtcaaacacccaatacatgaatatgtggggtggacataagttggagttcagaaccattggtggaactttgaatatctctacccaaggttcaaccaacacttctattaatcctgttacacttccattcacctcaagagatgtttataggactgaatctttggctggtcttaacttgtttcttacacaacctgttaatggtgttcctagagttgatttccattggaaatttgttactcatcctatcgcttcagataacttctactacccaggatacgctggtatcggaacacaacttcaagattctgaaaacgagttgcctccagaggctaccggtcaacctaattatgaatcatactctcataggctttcacatattggattgatctcagcttctcatgttaaggctcttgtttattcatggacacataggtctgctgataggacaaacaccattgaacctaattctattacccaaatccctcttgttaaggctttcaatctttcttctggtgctgctgttgttagaggtcctggtttcactggtggagatattcttagaaggactaatacaggtactttcggagatattagggttaacatcaatcctccatttgctcaaagatatagggttagaatcaggtacgcttcaacaaccgatttgcaattccatacttctattaacggaaaggctatcaaccaaggaaatttttctgctactatgaatagaggtgaggatcttgattataagactttcaggacagttggtttcactacacctttctcatttttggatgttcaatctactttcacaattggtgcttggaacttttcatctggaaacgaagtttacatcgatagaatcgaatttgttcctgtggaggttacttatgaagctgagtacgatttcgaaaaggctcaagagaaagttaccgctctttttacctcaactaaccctagaggtcttaagactgatgttaaagattaccatatcgatcaagtttctaatcttgttgaatcattgtctgatgagttttaccttgatgaaaagagggaattgtttgaaatcgtgaagtatgccaagcaactccacattgaaagaaatatgtaa

SEQ ID NO: 13 - DNA, 2028 bp, Artificial Sequence
  Description of Artificial Sequence Synthetic FBG cotton nucleotide motif
atgaacccttatcagaacaagaacgagtatgagatttttaacgctccatccaacggctttagcaagtcaaacaactacagcaggtatccacttgctaacaagcctaatcaaccattgaagaataccaactacaaggattggcttaatgtttgccaagataatcaacaatatggtaacaatgctggaaacttcgcttcttcagagacaattgttggtgtttctgctggtatcatcgttgttggtacaatgcttggtgcttttgctgctcctgttttggctgctggtattatttcttttggaacattgcttcctattttctggcaaggttctgatcctgctaacgtttggcaagatttgttgaatatcggtggtaggccaatccaagagattgataagaatatcattaacgttttgacctctatcgttacacctattaagaaccaacttgataagtaccaagaatttttcgataaatgggaacctgctaggacacatgctaacgctaaggctgttcatgatcttttcaccaccttggagccaattatcgataaggatttggatatgcttaagaacaacgcttcatacagaatccctactcttccagcttatgctcaaattgctacatggcatcttaatttgcttaagcatgctgctacttattacaatatctggcttcaaaaccaaggaatcaatccatcaacttttaattcatctaactactatcaaggttatcttaagaggaaaattcaagagtatactgattactgcattcaaacttataacgctggacttaccatgattaggacaaataccaacgctacttggaatatgtataatacctacaggcttgagatgactttgaccgttttggatcttatcgctatcttcccaaattatgatccagagaagtaccctatcggtgttaagtcagaacttattagagaagtttacactaatgttaactctgatacttttagaacaatcacagagttggaaaacggtcttacaagaaatccaacactttttacctggatcaaccaaggaagattctacactaggaactctagggatattcttgatccttacgatattttctcattcacaggaaatcaaatggcttttactcatacaaacgatgataggaatatcatctggggtgctgttcatggaaacatcatctcacaagatacatcaaaggttttcccattttacaggaacaagcctatcgataaggttgagatcgttaggcatagggagtattctgatattatctatgagatgatcttcttttcaaactcatctgaagttttcagatattcatctaactctactatcgaaaacaattacaaaagaacagattcttatatgattcctaagcaaacttggaagaacaaggaatacggacatactctttcttatatcaagacagataactacatcttctcagttgttagagagaggagaagggttgctttctcatggactcatacttctgttgattttcaaaacactattgatttggataacattacccaaattcatgctcttaaggctttgaaggtttcttcagattctaaaatcgttaagggtcctggacatactggaggagatttggttattttgaaggattctatggattttagagttagatttcttaagaacgtttcaaggcaataccaagttaggattagatatgctaccaacgctcctaagacaactgttttcttgactggtattgatacaatctcagttgaacttccatctacaacctctaggcaaaatccaaatgctactgatcttacttacgctgatttcggatacgttacctttccaagaaccgttcctaacaaaactttcgagggtgaagatacacttttgatgacattgtatggtacacctaaccattcttataacatctacattgataagatcgagtttatcccaattactcaatctgttttggattatacagaaaagcagaatatcgagaagacccagaagattgtgaatgatttgtttgtgaactga

SEQ ID NO: 14 - DNA, 7568 bp, Artificial Sequence
  Description of Artificial Sequence Synthetic FBG cotton nucleotide motif
cccgggtcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaacacgctggaattctagtatactaaaccatgttgattagggatgctgttcctggagatttgcctggtattcttgaaatccataatgaggctattgctaactctactgctatctgggatgaaacacctgctgatttggatgagagaaggagatggttcgatgataggagagctaatggttttccagttcttgttgctgatgttgatggtgttgttgctggatatgcttcttacggagtttggagggctaagtcttcatacagacatactgttgaaaactcagtttacgttcatgttgatcatcataggagaggtattgctaccgctttgatgactgaacttatcgagagggctagagctggtggtattcatgttatcgttgcttctgttgaatcaactaatgctacatcagttgctttgcatgagaggttcggtttcagaatcgttgctcacatgcctgaggttggtaggaaattcggaagatggcttgatatgacatatcttcaattgaccctttgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccctcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaacacgctggaattctagtatactaaaccatgaaactcaaaaaccaagacaaacatcagagcttctcctcaaacgctaaggtggataaaatcagcacagacagcctcaaaaacgagactgatattgagcttcaaaacatcaatcatgaagattgtttgaagatgtcagaatatgagaacgttgagcctttcgtttcagcttctacaattcaaaccggtattggaatcgctggaaagattcttggtacacttggagttcctttcgctggacaagttgcttcactttactcttttattttgggtgaactttggccaaagggaaaaaatcaatgggaaatttttatggagcatgttgaagagattatcaaccaaaagatttctacttatgctagaaataaggctttgactgatcttaaaggtttgggagatgctcttgctgtttaccatgattcattggagtcttgggttggtaacagaaataacacaagagctaggtctgttgttaggtcacaatatattgctcttgaattgatgttcgttcaaaaattgccatcttttgctgtttcaggagaagaggttcctcttttgccaatctacgctcaagctgctaaccttcatcttttgcttttgagggatgcttcaattttcggaaaggagtggggtctttcttcttctgaaatctctactttttacaatagacaagttgagagggctggagattattcagatcattgcgttaaatggtactctaccggtcttaataacttgagaggaactaacgctgaatcatgggttaggtacaatcaattcagaagggatatgactcttatggttcttgatttggttgctttgttcccttcttatgatacacaaatgtacccaattaagactacagctcaacttactagagaggtttatacagatgctatcggaaccgttcatcctcatccatcattcacttctaccacttggtacaataacaattttctgctattgaggctgctgttgttaggaacccacatcttttggatttcttggaacaagttacaatctattctcttctttctaggtggtcaaacacccaatacatgaatatgtggggtggacataagttggagttcagaaccattggtggaactttgaatatctctacccaaggttcaaccaacacttctattaatcctgttacacttccattcacctcaagagatgtttataggactgaatctttggctggtcttaacttgtttcttacacaacctgttaatggtgttcctagagttgatttccattggaaatttgttactcatcctatcgcttcagataacttctactacccaggatacgctggtatcggaacacaacttcaagattctgaaaacgagttgcctccagaggctaccggtcaacctaattatgaatcatactctcataggctttcacatattggattgatctcagcttctcatgttaaggctcttgtttattcatggacacataggtctgctgataggacaaacaccattgaacctaattctattacccaaatccctcttgttaaggctttcaatctttcttctggtgctgctgttgttagaggtcctggtttcactggtggagatattcttagaaggactaatacaggtactttcggagatattagggttaacatcaatcctccatttgctcaaagatatagggttagaatcaggtacgcttcaacaaccgatttgcaattccatacttctattaacggaaaggctatcaaccaaggaaatttttctgctactatgaatagaggtgaggatcttgattataagactttcaggacagttggtttcactacacctttctcatttttggatgttcaatctactttcacaattggtgcttggaacttttcatctggaaacgaagtttacatcgatagaatcgaatttgttcctgtggaggttacttatgaagctgagtacgatttcgaaaaggctcaagagaaagttaccgctctttttacctcaactaaccctagaggtcttaagactgatgttaaagattaccatatcgatcaagtttctaatcttgttgaatcattgtctgatgagttttaccttgatgaaaagagggaattgtttgaaatcgtgaagtatgccaagcaactccacattgaaagaaatatgtaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctcgcgatcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaacacgctggaattctagtatactaaaccatgaacccttatcagaacaagaacgagtatgagatttttaacgctccatccaacggctttagcaagtcaaacaactacagcaggtatccacttgctaacaagcctaatcaaccattgaagaataccaactacaaggattggcttaatgtttgccaagataatcaacaatatggtaacaatgctggaaacttcgcttcttcagagacaattgttggtgtttctgctggtatcatcgttgttggtacaatgcttggtgcttttgctgctcctgttttggctgctggtattatttcttttggaacattgcttcctattttctggcaaggttctgatcctgctaacgtttggcaagatttgttgaatatcggtggtaggccaatccaagagattgataagaatatcattaacgttttgacctctatcgttacacctattaagaaccaacttgataagtaccaagaatttttcgataaatgggaacctgctaggacacatgctaacgctaaggctgttcatgatcttttcaccaccttggagccaattatcgataaggatttggatatgcttaagaacaacgcttcatacagaatccctactcttccagcttatgctcaaattgctacatggcatcttaatttgcttaagcatgctgctacttattacaatatctggcttcaaaaccaaggaatcaatccatcaacttttaattcatctaactactatcaaggttatcttaagaggaaaattcaagagtatactgattactgcattcaaacttataacgctggacttaccatgattaggacaaataccaacgctacttggaatatgtataatacctacaggcttgagatgactttgaccgttttggatcttatcgctatcttcccaaattatgatccagagaagtaccctatcggtgttaagtcagaacttattagagaagtttacactaatgttaactctgatacttttagaacaatcacagagttggaaaacggtcttacaagaaatccaacactttttacctggatcaaccaaggaagattctacactaggaactctagggatattcttgatccttacgatattttctcattcacaggaaatcaaatggcttttactcatacaaacgatgataggaatatcatctggggtgctgttcatggaaacatcatctcacaagatacatcaaaggttttcccattttacaggaacaagcctatcgataaggttgagatcgttaggcatagggagtattctgatattatctatgagatgatcttcttttcaaactcatctgaagttttcagatattcatctaactctactatcgaaaacaattacaaaagaacagattcttatatgattcctaagcaaacttggaagaacaaggaatacggacatactctttcttatatcaagacagataactacatcttctcagttgttagagagaggagaagggttgctttctcatggactcatacttctgttgattttcaaaacactattgatttggataacattacccaaattcatgctcttaaggctttgaaggtttcttcagattctaaaatcgttaagggtcctggacatactggaggagatttggttattttgaaggattctatggattttagagttagatttcttaagaacgtttcaaggcaataccaagttaggattagatatgctaccaacgctcctaagacaactgttttcttgactggtattgatacaatctcagttgaacttccatctacaacctctaggcaaaatccaaatgctactgatcttacttacgctgatttcggatacgttacctttccaagaaccgttcctaacaaaactttcgagggtgaagatacacttttgatgacattgtatggtacacctaaccattcttataacatctacattgataagatcgagtttatcccaattactcaatctgttttggattatacagaaaagcagaatatcgagaagacccagaagattgtgaatgatttgtttgtgaactgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtacc

SEQ ID NO: 15 - DNA, 489 bp, Artificial Sequence; identical to SEQ ID NO: 11
  Description of Artificial Sequence Synthetic FBG cotton nucleotide motif
  CDS (1)..(486)

SEQ ID NO: 16 - DNA, 2160 bp, Artificial Sequence; identical to SEQ ID NO: 12
  Description of Artificial Sequence Synthetic FBG cotton nucleotide motif
  CDS (1)..(2157)

SEQ ID NO: 17 - DNA, 2028 bp, Artificial Sequence; identical to SEQ ID NO: 13
  Description of Artificial Sequence Synthetic FBG cotton nucleotide motif
  CDS (1)..(2025)

SEQ ID NO: 18 - DNA, 712 bp, Artificial Sequence
  Description of Artificial Sequence Synthetic FBG cotton nucleotide motif
cccgggtcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaacacgctggaattctagtatactaaacc

SEQ ID NO: 19 - DNA, 537 bp, Artificial Sequence
  Description of Artificial Sequence Synthetic insert sequence and Gossypium hirsutum
cccagaagattgtgaatgatttgtttgtgaactgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtaccgtaaacctaagagaaaagagcgtttacagctgtgacaggaatcttggctggttcaaatcgatccgcctcacttgaagacacccagcgttctatccccatcgggacgttggccgccacacttactactactggactctatttagtctccgtcttactttttggagctgttgccaccagagacaagcttgactgacaggctacttacggctacaattgcttggccttttccagctatcattcata

SEQ ID NO: 20 - DNA, 24 bp, Artificial Sequence
  Description of Artificial Sequence Synthetic primer sequence from inserted DNA
cccagaagattgtgaatgatttgt

SEQ ID NO: 21 - DNA, 24 bp, Artificial Sequence
  Description of Artificial Sequence Synthetic primer sequence from Gossypium hirsutum Genome
tatgaatgatagctggaaaaggcc

SEQ ID NO: 22 - protein, 162 aa, Artificial Sequence
  Description of Artificial Sequence Synthetic FBG cotton motif
MLIRDAVPGDLPGILEIHNEAIANSTAIWDETPADLDERRRWFDDRRANGFPVLVADVDGVVAGYASYGVWRAKSSYRHTVENSVYVHVDHHRRGIATALMTELIERARAGGIHVIVASVESTNATSVALHERFGFRIVAHMPEVGRKFGRWLDMTYLQLTL

SEQ ID NO: 23 - protein, 719 aa, Artificial Sequence
  Description of Artificial Sequence Synthetic FBG cotton motif
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVRSQYIALELMFVQKLPSFAVSGEEVPLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNM

SEQ ID NO: 24 - protein, 675 aa, Artificial Sequence
  Description of Artificial Sequence Synthetic FBG cotton motif
MNPYQNKNEYEIFNAPSNGFSKSNNYSRYPLANKPNQPLKNTNYKDWLNVCQDNQQYGNNAGNFASSETIVGVSAGIIVVGTMLGAFAAPVLAAGIISFGTLLPIFWQGSDPANVWQDLLNIGGRPIQEIDKNIINVLTSIVTPIKNQLDKYQEFFDKWEPARTHANAKAVHDLFTTLEPIIDKDLDMLKNNASYRIPTLPAYAQIATWHLNLLKHAATYYNIWLQNQGINPSTFNSSNYYQGYLKRKIQEYTDYCIQTYNAGLTMIRTNTNATWNMYNTYRLEMTLTVLDLIAIFPNYDPEKYPIGVKSELIREVYTNVNSDTFRTITELENGLTRNPTLFTWINQGRFYTRNSRDILDPYDIFSFTGNQMAFTHTNDDRNIIWGAVHGNIISQDTSKVFPFYRNKPIDKVEIVRHREYSDIIYEMIFFSNSSEVFRYSSNSTIENNYKRTDSYMIPKQTWKNKEYGHTLSYIKTDNYIFSVVRERRRVAFSWTHTSVDFQNTIDLDNITQIHALKALKVSSDSKIVKGPGHTGGDLVILKDSMDFRVRFLKNVSRQYQVRIRYATNAPKTTVFLTGIDTISVELPSTTSRQNPNATDLTYADFGYVTFPRTVPNKTFEGEDTLLMTLYGTPNHSYNIYIDKIEFIPITQSVLDYTEKQNIEKTQKIVNDLFVN